(19) United States
(12) Patent Application Publication
(10) Pub. No.: US 2011/0021626 A1
(43) Pub. Date: Jan. 27, 2011

(54) ENCAPSULATION OF VITAMIN C INTO WATER SOLUBLE DENDRIMERS

(75) Inventors: Didier Astruc, Pessac (FR); Jaime Ruiz Aranzaes, Begles (FR); Élodie Boisselier, Talence (FR)

Correspondence Address: ARENT FOX LLP, 1050 CONNECTICUT AVENUE, N.W., SUITE 400, WASHINGTON, DC, 20036, US

(73) Assignee: CENTRE NATIONAL DE LA RECHERCHE SCIENTIFIQUE - CNRS, Paris Cedex 16 (FR)

(21) Appl. No.: 12/863,877

(22) PCT Filed: Jan. 21, 2009
(86) PCT No.: PCT/FR09/00064

§ 371 Date: Sep. 23, 2010

(30) Foreign Application Priority Data
Jan. 21, 2008 (FR) 0850372

(51) Int. Cl. A61K 31/341; C07D 307/62; C07F 7/02; A61P 17/00

(52) U.S. Cl. 514/474; 549/315; 549/214

(57) ABSTRACT

The invention relates to a conjugated dendrimer that comprises at least one water-soluble dendrimer and at least one vitamin C molecule. The conjugated dendrimer can be used e.g. for preparing cosmetic or pharmaceutical compositions. The dendrimers have a good vitamin C load capacity and are biocompatible.

### TECHNICAL FIELD

The present invention relates to the encapsulation of vitamin C using water-soluble dendrimers, and also, in particular, to its method of preparation.

The use of this dendrimer that encapsulates vitamin C relates, for example, to the fields of pharmacy, cosmetics, organic chemistry, and also to that of green chemistry.

In the description below, the references between parentheses (X) refer to the list of references presented after the examples.

### PRIOR ART

As its name indicates, a dendrimer is a molecule, the architecture of which echoes that of the branches of a tree. Specifically, it is a macromolecule of three-dimensional structure, similar to a hyperbranched polymer, where the branched monomers are associated according to an arborescent process around a multivalent central core.

Dendrimers generally adopt a highly branched and multifunctionalized, very regular or spherical globular shape. They are constituted of three specific regions:
a multivalent central core;
a defined number (constituting the multivalency) of intermediate dendritic branches connected to the multivalent central core where each dendritic branch is constituted of a certain branching generation number; and
the periphery constituted of a multitude of functional terminal groups.

These molecules have both internal cavities and a large number of readily accessible terminal groups at the periphery, which may be responsible for very varied properties and reactivities.

Mention may be made, for example, of the document by Tomalia et al. Macromolecules (1986) 19, page 2466 (1) which describes the construction of the first water-soluble dendrimers.

Dendrimers are constructed step by step using a succession of sequences, each resulting in a new generation. Structural control is a determining factor for the specific properties of these macromolecules.

Dendrimers generally adopt a highly branched and multifunctionalized, very regular or spherical globular shape. Moreover, dendrimers are generally monodispersed, unlike hyperbranched polymers. Obtaining these regular structures, which the dendrimers are, requires synthesis methods other than standard polymerizations. Specifically, the synthesis of dendrimers is difficult since it requires numerous steps in order to protect the active site, which is an obstacle to the synthesis of large amounts. Furthermore, obtaining dendrimers with multiple arborescences that are defined and/or functionalized in a specific manner is a real challenge for chemists since it requires quantitative and appropriate reactions in order to avoid mixtures on the branches. By way of comparison, hyperbranched polymers, the molecular architecture of which is irregular, are obtained by polymerization of multifunctional branched monomers following a disordered and unlimited process.

Thus, these macromolecules are adjustable: their structure, their flexibility, their porosity and their morphology may be optimized in order to obtain the desired properties. The control of the architecture of the dendrimers and the chemical nature of the terminal branches which may thus provide various functions give rise to a great interest for many very promising applications, especially in the field of medicine as described, for example, in the article by Astruc, C.R. Acad. Sci. (1996), 322, Série 2b, pages 757-766 (2), but also in the following fields: agri-food, flavors, paints, catalysis, decontamination, gene therapy, photodynamic therapy, biological sensors, nanoelectronics, etc.

Moreover, the vectoring of active molecules is today a major issue in the pharmaceutical and cosmetic fields, as described, for example, in the publication by Vandamme et al., J. Control. Release (2005), 102(1), pages 23-38 (3). It corresponds to the transport of active molecules to an active site which makes it possible to adjust the pharmacokinetics and biodistribution of these molecules by favoring their presence at active sites while limiting their toxicity or impact on healthy tissues. Furthermore, vectoring enables a protection of the active molecules with respect to the medium in which they are introduced by avoiding, for example, their metabolization or their degradation, as described, for example, in the publication by Zhuo et al. J. Control. Release (1999) 57, page 249 (4).

Vitamins are of great interest, in particular for industrialists in the pharmaceutical and cosmetic fields. Specifically, they are involved in numerous biological and physiological processes and are essential for the body. By way of example, vitamin C, or ascorbic acid (AA) has many properties. Known for its tonifying and anti-fatigue actions, it is involved, for example, in the synthesis of red blood cells and contributes to the immune system. Thus, it participates in the defenses of the body and thus plays a role in the battle against infection. Vitamin C also promotes the absorption of iron, which makes it possible to prevent the onset of anemia. It is also required in synthesis of collagen of the skin and is essential to wound healing. It is also a powerful free-radical scavenger and antioxidant which makes it possible to protect the body from free radicals, and to prevent oxidative degradation and aging. Thus, these applications, especially cosmetic applications, are numerous, also stimulating cell metabolism and having a photoprotective effect.

However, some vitamins, and especially vitamin C, cannot be synthesized by the human body, hence the need for an exogenous supply.

Moreover, vitamins are fragile compounds, easily degraded by light, heat, oxygen from the air, etc. In particular, vitamin C, the most fragile among all the vitamins, is not very stable in solution and is rapidly eliminated by the body. Existing pharmaceutical and cosmetic products therefore have a limited usage time. To overcome this problem of instability, they use a very different formulation which does not allow a modulated release. Furthermore, since this molecule is not currently vectored, its dispersion is uncontrolled and non-specific.

There is therefore a real need to have novel compounds that make it possible to vector and to control the release of active molecules, in particular of cosmetic active agents and especially of vitamins, for example vitamin C.

Moreover, there is a real need to have biocompatible compounds, of low toxicity, of defined architecture, of controlled size and shape, of high loading capacity, and of specific functionalization in order to optimize the encapsulation of vitamin C.

Moreover, there is a real need for a method that makes it possible to encapsulate vitamin C with good efficiencies, to protect the environment and to reduce the costs of obtaining these compounds.

### DESCRIPTION OF THE INVENTION

The objective of the present invention is specifically to respond to these needs and drawbacks of the prior art by providing a conjugated dendrimer comprising at least one water-soluble dendrimer encapsulating at least one molecule of vitamin C.

The expression “conjugated dendrimer” is understood, within the meaning of the present invention, to mean the association of at least one water-soluble dendrimer with at least one molecule of vitamin C. Moreover, one molecule of vitamin C may be associated with one or more dendrimers which may be identical or different, for example if the PEG chains of various dendrimers participate in the stabilization of the molecules of vitamin C.

The expression “water-soluble dendrimer” is understood, within the meaning of the present invention, to mean a dendrimer which can be dissolved in water, that is to say the aqueous solution of which is clear. In particular, the NMR spectrum of this solution makes it possible to visualize all the groups of the dendrimer present. It may be, for example, dendrimers that have, at their surface, functional groups that are hydrophilic and/or not very hydrophobic, that is to say polar functional groups and/or functional groups capable of creating hydrogen bonds with the water molecules. It may be, for example, dendrimers that have, at their surface, amino (—NH₂, —NH— or —N—), ammonium (—NH₄⁺), cyano (—CN), amido (—C(═O)—NH₂ or —C(═O)—NH—), hydroxyl (—OH), alcoholates (—O⁻), carboxyl (—C(═O)—OH), carboxylate (—C(═O)—O⁻), carbonyl (—C(═O)H or —C(═O)—), oxy (—O—) or ester (—C(═O)—O—) functional groups, preferably amino, ammonium, amido and oxy functional groups.

The expression “to encapsulate” is understood, within the meaning of the present invention, to mean the fact of surrounding a molecule, for example in the form of a capsule or a shell, making it possible to isolate it, to stabilize it and/or to protect it from the external medium. This encapsulation may take place at the core but also at the periphery of the dendrimer where the molecules of vitamin C are turned toward the inside of the dendrimer and are thus stabilized and protected.

The term “association” is understood, within the meaning of the present invention, to mean the assembly of two or more different molecules, for example by supramolecular bonds. The molecules of vitamin C may, for example, be associated on the inside of the dendrimer or at the surface thereof.

The expression “supramolecular bond” is understood, within the meaning of the present invention, to mean the assembling of several different molecules by hydrogen bonds, ionic bonds, coordination bonds and/or by hydrophobic interactions.

Surprisingly, the inventors have demonstrated that the molecules of vitamin C were encapsulated by the dendrimers of the present invention and did not remain in solution. In other words, the molecules of vitamin C are associated either with the core, or with the periphery of the dendrimers of the present invention. The dendrimers of the present invention thus make it possible to stabilize the molecules of vitamin C and/or to protect them from the external medium. Indeed, the molecules of vitamin C are incorporated into a “capsule” protected by a hydrophilic crown.

Furthermore, the dendrimers have the advantage of being unique molecules (that is to say having a polymolecularity equal to one), thus are perfectly defined, with exact chemical formulae, and which may be characterized very precisely (for example by proton and/or carbon nuclear magnetic resonance (NMR) techniques, microanalysis, infrared spectrometry, or else mass spectrometry). Dendrimers are therefore suitable molecules for biological applications which require high purity and great knowledge of the molecules introduced into the biological medium, for example of their method of assembly, operation, stability or the way in which they could be metabolized. Indeed, biological applications are much more difficult to carry out in the case of polydisperse or polymolecular molecules such as, for example, hyperbranched polymers. In particular, hyperbranched polymers comprise a mixture of hyperbranched molecules which do not have the same molecular weight and will not stabilize an identical number of biological molecules for each of them, which generates much more uncertainty and many more difficulties for adapting them to a biological use.

Moreover, the water-soluble dendrimers according to the present invention seem to be suitable vectors for the transport of molecules of vitamin C, that is to say of ascorbic acid. Indeed, the controlled multivalency of the dendrimers of the present invention may be used to attach one or more targeting and solubilizing groups and/or substances to the periphery. This attachment or association may be carried out by supramolecular bonds which enable molecules of vitamin C to be held at the core and at the periphery of the dendrimers.

The dendrimers of the present invention may behave as vectors of vitamin C. Vectoring aims to modify the stability and the pharmacokinetic properties of the active principles transported such as the crossing of anatomical, physiological barriers, and the targeting thereof. The particular features of the dendrimers, and their globular shape make these novel molecular architectures ideal transporters of loaded molecules. Indeed, these molecules may be used in a large number of applications including controlling the release of pharmaceutical or else cosmetic products.

According to one particular embodiment of the invention, the water-soluble dendrimer may have a symmetrical radial structure.

Indeed, the inventors have produced dendrimers that are said to be “perfect”. The expression “perfect dendrimer” is understood, within the meaning of the present invention, to mean a dendrimer of symmetrical structure which is defect-free, with a complete and uniform functionalization of the branches. The high symmetry of the architecture of the dendrimers was verified by various characterization methods (NMR, mass, etc.). This data is given in the section “Examples”.

The dendrimers of symmetrical structure have the advantage of being able to be perfectly controlled and characterized, with reproducible results, which is essential for an industrial scale-up. However, “imperfect” dendrimers may also encapsulate molecules of vitamin C. The expression “imperfect dendrimer” is understood, within the meaning of the present invention, to mean a dendrimer which does not come under the definition of the dendrimer said to be “perfect”.

Moreover, according to the invention, the water-soluble dendrimer may be constituted of g generations, g being an integer ranging from 0 to 10, preferably from 0 to 4, in order to respect a size that can be admitted into the body.

The term “generation” is understood, within the meaning of the present invention, to mean a repetition of branching units or monomers organized in a layer around the central core. A generation is counted from each division of a branch into at least two branches. The generation number g of the dendrimer corresponds to the number of concentric monomer layers, but may also correspond to the number of sequences necessary for the synthesis of the dendrimer.

The generation number g of the dendrimer leads to a precise size of the dendrimer and also plays an important role in the conformation of the dendrimer.

Advantageously, the water-soluble dendrimer may have a globular structure. It may be, for example, a lobe-shaped structure, an elliptical structure, a spherical structure, which is perfect or imperfect, preferably a perfect spherical structure.

Indeed, dendrimers have a dendritic topology, that is to say an architecture constructed according to an arborescent process around a multifunctional central core, analogically to a dendritic neural network. The branched units are repeated monomers organized in layers, also known as generations. Depending on the dendrimers, beyond a certain number of generations, they may adopt a globular form if the molecular segments are flexible.

The dendrimers of globular structure have the advantage of being able to accommodate molecules within their cavities with a greater capacity. Indeed, the dendritic topology establishes dynamic cavities that are absent in a linear polymer for example, the topology of which is different. The term “cavities” is understood within the meaning of the present invention to mean the spaces present between the branches of the dendrimers. Since a dendrimer is not set in solution, the cavities may be dynamic due to the movement of the branches within the solution, guided by Brownian motion.

By way of example, the following comparative table 1 gives details of the various properties of a linear polymer and of a dendrimer. Thus, depending on the desired criteria, the dendrimer may be preferred in order to obtain the various properties below which differ from those of a linear polymer.

TABLE 1

| | LINEAR POLYMER | DENDRIMER |
|---|---|---|
| Shape | spherical ball | globular |
| Viscosity | low | high |
| Crystallinity | high | amorphous |
| Reactivity | low | high |
| Compressibility | high | low |
| Structural control | low | very high |

In particular, the main advantage of a globular dendrimer compared to a linear polymer is the size of its internal cavities which may be optimized in order to accommodate large molecules. Indeed, in the case of the dendrimer, the cavities may be wide and organized so as to allow greater accommodation or encapsulation capacities. On the other hand, in a linear polymer folded up into a spherical ball, the structure is random and the cavities are not optimized.

According to another particular embodiment of the invention, the water-soluble dendrimer may have a diameter of less than 200 Å, preferably of 10 Å to 50 Å. This diameter depends on the generation of the dendrimer.

According to the invention, the water-soluble dendrimer may have a molecular weight ranging from 20 to 50 000 g/mol, for example from 500 to 15 000 g/mol, for example from 773 to 7168 g/mol.

The water-soluble dendrimer of the present invention may be, for example, a dendrimer of formula (I) below:

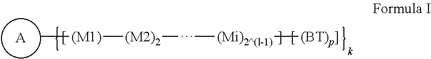

in which:
(a) A is the central core of the dendrimer, of multivalency k, where:
   k is an integer ranging from 2 to 9;
   A is a radical chosen from the group comprising a C₁ to C₂₂ heteroalkyl, a phenyl optionally substituted by one or more C₁ to C₆₀ heteroalkyl groups, the radical A possibly optionally comprising one or more carbons substituted by an oxo (═O) group;
(b) Mi is a monomer of generation i, where:
   i is an integer ranging from 0 to g, g being the generation number of the dendrimer;
   when i=0, Mi is nonexistent, the terminal branch BT is then directly connected to the central core A;
   when i>0, Mi is a C₁ to C₂₂ heteroalkyl radical, in which one or more carbons may optionally be substituted by an oxo (═O) group, for example Mi may be a —(CH₂)ₐ—N* radical or a —(CH₂)ₐ—C(═O)—NH—(CH₂)_{b}—N* radical where a and b are integers from 1 to 6 and the symbol * denotes the point of attachment of the Mi radical to/with the monomer of higher generation;
(c) BT is the terminal branch, and p the number of terminal units derived from the monomer of lower generation where:
   p is an integer ranging from 1 to 3, preferably p is 2 or 3, preferably p is 3;
   BT is a radical chosen from the group comprising a hydrogen, an —NH₂ group, an ammonium group, an —OH group, a C₁ to C₂₂ heteroalkyl, a phenyl substituted by one or more C₁ to C₆₀ heteroalkyl groups, the radical BT possibly optionally comprising one or more carbons substituted by an oxo (═O) group; for example, BT may be a —C(═O)—CH₂—O—(CH₂)₂—O—(CH₃) radical or a —C(═O)—Ph((OC₂H₄)₃—OCH₃)₃ radical.

The term “multivalency” is understood, within the meaning of the present invention, to mean the number of dendritic branches connected to the central core of the dendrimer.

The expression “dendritic branch” is understood, within the meaning of the present invention, to mean a branched branch. In particular, it may be constituted of a chain of monomers Mi, which are identical or different, where each branching between the monomers divides into two or more branches. For example, in the formula (I), the dendritic branch may correspond to: [(M1)−(M²)₂− . . . −(Mi)_{2̂(i-1)}]−[(BT)ₚ].

The expression “terminal branch” is understood, within the meaning of the present invention, to mean the part of the dendritic branch constituted by the terminal unit located at the periphery of the dendrimer. It may be, for example, a monomer of last generation g of the dendrimer, a hydrophilic functional group, a dendron, etc. For example, in the formula (I), the terminal branch corresponds to: (BT).

The expression “intermediate dendritic branch” is understood, within the meaning of the present invention, to mean the part of the dendritic branch connecting the multivalent central core A to the terminal branch BT. For example, in the formula (I), the intermediate dendritic branch corresponds to: [(M1)−(M2)₂− . . . −(Mi)_{2̂(i-1)}].

The term “dendron” is understood, within the meaning of the present invention, to mean a branched dendritic branch (for example which is not terminal or internal), for example comprising two or more branchings. For example, the dendron may be a hyperbranched dendritic branch, that is to say comprising three or more branchings. As a dendron, mention may, for example, be made of pentafluorophenyl tris 3,4,5-tri(triethyleneoxy)benzoate (or pentafluorophenyl tris 3,4,5-tri(triethylene glycol)benzoate or PFPTTEG).

The term “alkyl” is understood, within the meaning of the present invention, to mean an optionally substituted, saturated or unsaturated, linear, cyclic or branched, carbon-based radical comprising 1 to 60 carbon atoms, for example 1 to 57 carbon atoms, for example 1 to 28 carbon atoms, for example 1 to 22 carbon atoms, for example 1 to 6 carbon atoms.

The term “alkene” is understood, within the meaning of the present invention, to mean an alkyl radical, as defined previously, having at least one carbon-carbon double bond.

The term “alkyne” is understood, within the meaning of the present invention, to mean an alkyl radical, as defined previously, having at least one carbon-carbon triple bond.

The term “aryl” is understood, within the meaning of the present invention, to mean an aromatic system comprising at least one ring that satisfies Hückel's rule of aromaticity. Said aryl is optionally substituted, may be monocyclic or polycyclic and may comprise from 6 to 10 carbon atoms.

The term “heteroalkyl” is understood, within the meaning of the present invention, to mean an alkyl radical as defined previously, said alkyl system comprising at least one heteroatom, in particular chosen from the group comprising sulfur, oxygen, nitrogen and silicon.

According to the invention, the water-soluble dendrimer may have a hydrophobic central core and a hydrophilic crown. It is understood that the hydrophobic core does not necessarily comprise only hydrophobic groups. In particular, the hydrophobic core may contain heteroatoms (for example N, O, S and/or Si). In this sense, “hydrophobic core” signifies a core that has a hydrophobic nature with respect to the periphery of the dendrimer, which is hydrophilic. This hydrophobic core/hydrophilic crown system makes it possible, in particular to hold the molecules of vitamin C at the core of the dendrimer, thus preventing their contact with the external medium (or the biological environment) and therefore a premature oxidation of these molecules, while enabling the release of active molecules of vitamin C especially in biological medium.

According to one particular embodiment of the invention, the water-soluble dendrimer may have intermediate dendritic branches comprising heteroatoms, for example branches comprising amino (—NH— or —N—), amido (—C(═O)—NH—), oxy (—O—) or ester (—C(═O)—O—) groups, preferably amino groups, for example secondary or tertiary amine groups. Since these groups are situated within the dendrimer, this has the advantage of allowing a better encapsulation of molecules of vitamin C within the dendrimer, for example by hydrogen bonds, ionic bonds and/or coordination bonds, and thus prevent their contact with the external medium. Moreover, this also makes it possible to increase the number of molecules of vitamin C that are encapsulated.

According to one particular embodiment of the invention, the water-soluble dendrimer may have, at its periphery, mainly free primary amine functional groups or ammonium functional groups.

Indeed, the dendrimers comprising free primary amines or ammonium functional groups at the periphery have the advantage of being able to react either in a supramolecular manner with ascorbic acid (for example by forming ammonium ascorbates), as demonstrated in the examples section, or in a covalent manner with other chemical groups in order to modify the periphery of the dendrimer and therefore its properties.

For example, they may be dendrimers comprising a diaminobutane (DAB) core and poly(propylene)imine) (PPI) branches, such as for example the dendrimers depicted in FIGS. 1 to 4, or poly(amido)amine (PAMAM) dendrimers, such as for example the dendrimers depicted in FIGS. 5 to 9.

Thus, according to one particular embodiment of the invention, the water-soluble dendrimer may have one of the following structures: DAB G2 (FIG. 1), DAB G3 (FIG. 2), DAB G4 (FIG. 3), DAB G5 (FIG. 4), PAMAM G0 (FIG. 5), PAMAM G1 (FIG. 6), PAMAM G2 (FIG. 7), PAMAM G3 (FIG. 8), PAMAM G4 (FIG. 9), etc.

Some of these dendrimers are commercial, for example the dendrimers DAB G2, DAB G3, DAB G5, PAMAM G1 and PAMAM G4 are sold by Sigma-Aldrich (Sigma-Aldrich Chemie S.a.r.l., L'Isle d'Abeau Chesnes, 38297 Saint-Quentin Fallavier, France), respectively under the references No. 679895 (DAB G2), No. 469076 (DAB G3), No. 469092 (DAB G5), No. 597414 (PAMAM G1) and No. 597856 (PAMAM G4).

Depending on the choice of dendrimers used, it is possible to promote the association of molecules of vitamin C preferably at the periphery then at the core of the dendrimers (such as, for example, in the case of PAMAM G4) or vice versa to promote firstly the association at the core.

According to one particular embodiment of the invention, the water-soluble dendrimer may be functionalized at its periphery with at least one organic surface agent.

The expression “surface agent” is understood according to the invention to mean a molecule present at the surface of the dendrimer that makes it possible to adjust its surface properties, for example:
to modify its solubility;
to adjust its toxicity and its biodistribution, for example in order to prevent its recognition by the reticuloendothelial system (“stealth” properties); and/or
to give it advantageous bioadhesion properties during oral, ocular, nasal administration; and/or
to permit it specific targeting of certain organs/tissues, etc.

According to one particular embodiment of the invention, the organic surface agent may be selected from the group comprising: a pharmaceutical molecule, a targeting molecule or a solubilizing group.

According to another particular embodiment of the invention, the organic surface agent may be a solubilizing group having polyethylene glycol chains.

Indeed, the functionalization of these dendrimers with various organic groups containing polyethylene glycol chains makes it possible to improve the solubility in water of these macromolecules as described in the document by Liu et al., J. Polym. Sci.: part A: Polym. Chem. (1999), page 3492 (5), and also the biocompatibility. Furthermore, surprisingly, in addition to these aforementioned important features for a product intended for a biological use, the dendrimers functionalized by polyethylene glycol chains have proved to be better molecular vectors via their encapsulation efficiency. Moreover, these molecular vectors may be modified at will so as to optimize the encapsulation of the molecules of vitamin C. Indeed, a dendron containing polyethylene glycol chains makes it possible not only to improve the degree of encapsulation of vitamin C molecules, but also to favor the encapsulation toward the inside of the dendrimer (for a better protection), for example by producing supramolecular bonds with the molecules of vitamin C, despite the steric hindrances which may occur. This property was unexpected, and was demonstrated by the inventors. By way of example, the dendrimer DAB G3 functionalized by the dendron PFPTTEG makes it possible to attach more than 150 molecules of vitamin C. Moreover, this dendrimer (with a hyperbranched dendron that therefore has a high steric hindrance) makes it possible to attach many more molecules of vitamin C than the dendrimer DAB G3 functionalized by the ligand MEAC. Other examples of encapsulation efficiencies of dendrimers according to the invention are given in the examples section.

According to one more particular embodiment of the invention, the organic surface agent may be a solubilizing group corresponding to one of the structures (II) or (III) below:

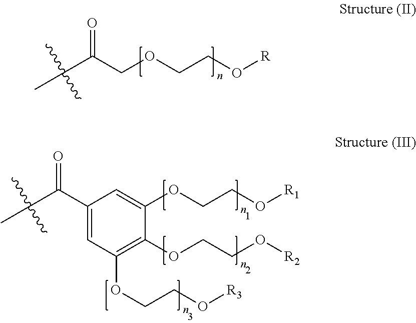

in which:
n, n₁, n₂ and n₃ independently represent an integer from 1 to 500; and
R, R₁, R₂ and R₃ independently represent a C₁ to C₆ alkyl, a C₂ to C₆ alkene, a C₂ to C₆ alkyne or a C₆ to C₁₀ aryl which is optionally substituted.

According to one more particular embodiment of the invention, the solubilizing group may correspond to one of the structures (IV) or (V) below:

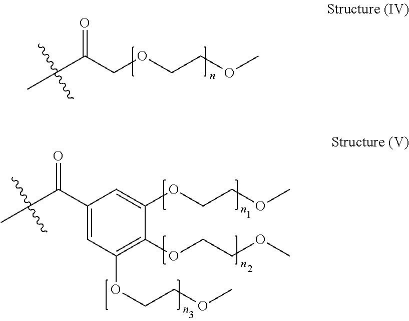

in which n, n₁, n₂ and n₃ represent 1 or 3, preferably, n represents 1 and n₁, n₂ and n₃ represent 3.

For example, the water-soluble dendrimer according to the invention may be a DAB-dendrimer or a PAMAM-dendrimer functionalized by the pentafluorophenyl tris 3,4,5-tri(triethyleneoxy)benzoate (or pentafluorophenyl tris 3,4,5-tri(triethylene glycol)benzoate or PFPTTEG) dendron or by the 2,2-(methoxyethoxy)acetyl chloride (MEAC) ligand. For example, it may be DAB G3 functionalized by the MEAC ligand (FIG. 10) or by the PFPTTEG dendron (FIG. 11).

According to one particular embodiment of the invention, the water-soluble dendrimer may be a nona-ammonium chloride corresponding to the following structure:

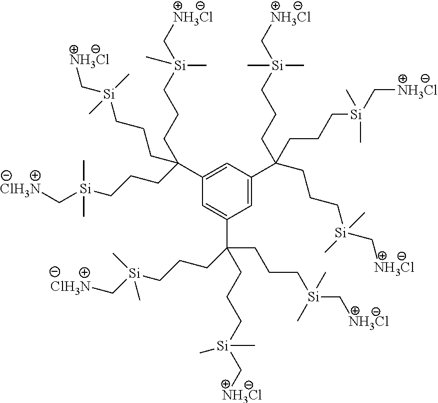

which dendrimer is functionalized at its periphery by a solubilizing group corresponding to one of the structures (II) or (III), preferably to the structure (III), below:

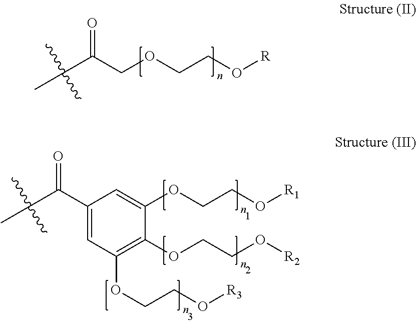

in which:
n, n₁, n₂ and n₃ independently represent an integer from 1 to 500; and
R, R₁, R₂ and R₃ independently represent a C₁ to C₆ alkyl, a C₂ ^{to C}₆ alkene, a C₂ to C₆ alkyne or a C₆ to C₁₀ aryl which is optionally substituted.

It should be noted that the nona-ammonium chloride dendrimer is not soluble in water, hence the interest in functionalizing it beforehand with solubilizing groups, for example with the PFPTTEG dendron, for the encapsulation of vitamin C. An example of functionalization methodology is given in the document by Pan et al., Macromolecules (2000), 33, pages 3731-3738 (6). Indeed, this document describes that it is preferable to functionalize the anionic dendrimers with solubilizing and biocompatible groups even if they have a lower toxicity than the cationic dendrimers as demonstrated in the document by Malik at al., J. Control. Release (2000), 65, page 133 (7).

Thus, according to one particular embodiment of the invention, the water-soluble dendrimer may correspond to the structure depicted in FIG. 12 (“nona-amine” dendrimer functionalized by the PFPTTEG dendron).

According to one particular embodiment of the invention, the water-soluble dendrimer may produce supramolecular bonds with the at least one molecule of vitamin C.

According to one particular embodiment of the invention, the at least one molecule of vitamin C may be held at the core or at the periphery of the at least one water-soluble dendrimer.

Indeed, their multiple cavities and their reactive groups at the periphery enable the recognition and the capture of anions, such as the ascorbate anion. Thus, vitamin C may be held in the core or held at the surface of the dendrimer by supramolecular bonding, and especially by electrostatic bonding. Indeed, the ascorbic acid molecules have a pKa equal to 4,11 at 25° C. in water, and thus form ammonium ascorbates with the amines of the dendrimers when both these products are in solution. Furthermore, the molecules of vitamin C also produce hydrogen bonds between their alcohol functional groups and the amines of the dendrimers. In the case of the functionalized dendrimers, the PEG groups may participate in the transport of the ascorbic acid molecules either by producing hydrogen bonds with the ascorbate molecules, or by holding them at the hydrophobic core of the dendrimer.

A dendrimer may contain up to 500 molecules of vitamin C. Thus, according to one particular embodiment of the invention, the average encapsulation number may be from 14 (±1) to 500 (±20) molecules of vitamin C per dendrimer. Moreover, this average encapsulation number may be adjusted as a function of the desired application. Indeed, depending on the amount of vitamin C added, it is possible to encapsulate it more or less up to a limit (which corresponds to the maximum number of molecules of vitamin C that can be encapsulated).

The release of vitamin C may take place in water, for example by exchange with water molecules, where the ascorbate molecules may first be protonated before being released in the form of ascorbic acid. The release may also take place in other solvents where vitamin C is soluble, such as for example ethanol. The release may be controlled by carrying out a dialysis in order to study the release kinetics. Indeed, the use of a porous semi-permeable membrane (the pores of which have identical and known diameters) makes it possible to separate the molecules of vitamin C from the dendrimers: via molecular stirring and osmosis effects, the small molecules, namely the molecules of vitamin C, pass through the membrane, while the large molecules, namely the dendrimers, are retained on the inside of the dialysis membrane. A quantification of these molecules of vitamin C makes it possible to monitor the release kinetics. Furthermore, the release kinetics of vitamin C can be modified depending on the pH, since the pH influences the ratio between the number of molecules of ascorbic acid and of ascorbate.

The invention also relates to a cosmetic composition comprising a conjugated dendrimer according to the invention.

Indeed, the conjugated dendrimer according to the invention may be formulated in a cosmetic product in order to ensure a modulated delivery of vitamin C to the skin for protective (free radical scavenging) and/or regenerating purposes.

Thus, the invention also relates to a conjugated dendrimer according to the invention as an antioxidant and/or anti-inflammatory agent.

Moreover, the invention also relates to the use of the conjugated dendrimer according to the invention for the preparation of a cosmetic composition, for example an anti-aging or anti-wrinkle composition.

The invention also relates to a cosmetic care method comprising a step that consists in applying a cosmetic composition comprising a conjugated dendrimer according to the invention to the skin.

The invention also relates to a method of preparing a conjugated dendrimer comprising at least one water-soluble dendrimer that encapsulates at least one molecule of vitamin C, said method comprising a step of reaction of said water-soluble dendrimer with said at least one molecule of vitamin C in a solvent and at a temperature which facilitate the formation of supramolecular bonds of the dendrimer and of the molecule of vitamin C. It should be noted that water is the solvent which enables both the solubility and the formation of bonds between the dendrimer and the vitamin C.

Indeed, bringing these two species together in a solvent instantaneously leads to the formation of supramolecular interactions.

According to one particular embodiment of the invention, during the preparation method, the association of the dendrimer and of the molecule of vitamin C may be carried out by means of at least one of the following bonds: formation of ammonium ascorbate with an amine functional group of the dendrimer, formation of a hydrogen bond between a hydroxyl functional group of the vitamin C and an amine functional group of the dendrimer.

According to the invention, the reaction step may be carried out in a solvent chosen from the group comprising water, R^{S}—OH alcohols where R^{S} is a C₁ to C₆ alkyl radical, for example ethanol, or a mixture thereof. Indeed, the solvent used may partially or completely solubilize the ascorbic acid and the dendrimer. For example, ascorbic acid is only partially soluble in ethanol.

According to the invention, the reaction step may be carried out for a reaction time of less than one minute, for example of less than 30 seconds, for example of less than one second. Indeed, the reaction time may range from a few milliseconds to a few seconds, preferably from a few milliseconds to one second.

According to the invention, the reaction step may be carried out at a temperature ranging from 3 to 90° C., preferably from 15 to 35° C., preferably from 20 to 30° C. Moreover, an increase of the temperature may make it possible to accelerate the rate of reaction.

According to the invention, the reaction step may be carried out at a pH ranging from 0 to 14, preferably at a pH from 6 to 8, preferably at a pH of 7. Moreover, the pH may influence the rate of reaction, but especially the encapsulation efficiency, indeed, the pH has an influence on the formation of hydrogen bonds between the ascorbic acid and the dendrimer.

According to one particular embodiment of the invention, the preparation method may comprise the reaction, in water, of a sufficient amount of vitamin C to obtain a concentration of less than 10² mg/ml, with a sufficient amount of dendrimer to obtain a conjugated dendrimer having a vitamin C/dendrimer ratio of 14 to 500, the reaction being carried out at a pH of 6 to 8, preferably 7, and at a temperature of 25° C.±10° C., preferably at a temperature of 25° C.±5° C.

Thus, the preparation method has the advantage of making use of green chemistry, the encapsulation possibly being entirely carried out in water. This has advantages both in terms of protecting the environment, and lower production cost, but also in terms of purity, enabling applications in many fields, including the pharmaceutical and cosmetic fields.

According to one particular embodiment of the invention, the preparation method may also comprise, before the reaction step, a functionalization step, which consists in functionalizing the periphery of the dendrimer with an organic surface agent selected from the group comprising: a pharmaceutical molecule, a targeting molecule or a solubilizing group.

Other advantages will also become apparent to a person skilled in the art reading the examples below, illustrated by the appended figures, which are given by way of illustration.

### BRIEF DESCRIPTION OF THE FIGURES

FIG. 1 represents the dendrimer DAB G2.

FIG. 2 represents the dendrimer DAB G3.

FIG. 3 represents the dendrimer DAB G4.

FIG. 4 represents the dendrimer DAB G5.

FIG. 5 represents the dendrimer PAMAM G0.

FIG. 6 represents the dendrimer PAMAM G1.

FIG. 7 represents the dendrimer PAMAM G2.

FIG. 8 represents the dendrimer PAMAM G3.

FIG. 9 represents the dendrimer PAMAM G4.

FIG. 10 represents the dendrimer DAB G3 functionalized by MEAC.

FIG. 11 represents the dendrimer DAB G3 functionalized by PFPTTEG.

FIG. 12 represents the nona-amine dendrimer functionalized by PFPTTEG.

FIG. 13 represents the graphs of the variation in the chemical shift δ (in ppm or parts per million) of the signals of the protons in ¹H NMR relative to the number N of molecules of ascorbic acid (AA) per dendrimer: (a) represents the graph of deshielding of the signals of the protons 1 to 4 of the dendrimer DAB G2 (in ppm) relative to N; and (b) the graph of shielding of the signals of the protons A to C of the AA (in ppm) relative to N.

FIG. 14 represents the graphs of the variation in the chemical shift δ (in ppm or parts per million) of the signals of the protons in ¹H NMR relative to the number N of molecules of ascorbic acid (AA) per dendrimer: (a) the graph of deshielding of the signals of the protons 1 to 4 of the dendrimer DAB G3 in ppm relative to N; and (b) the graph of shielding of the signals of the protons A to C of the AA in ppm relative to N.

FIG. 15 represents the graphs of the variation in the chemical shift δ (in ppm) of the signals of the protons in ¹H NMR relative to the number N of molecules of ascorbic acid (AA) per dendrimer: (a) the graph of deshielding of the signals of the protons 1 to 3 of the dendrimer DAB G5 in ppm relative to N; and (b) the graph of shielding of the signals of the protons A to C of the AA in ppm relative to N.

FIG. 16 represents the graphs of the variation in the chemical shift δ (in ppm) of the signals of the protons in ¹H NMR relative to the number N of molecules of ascorbic acid (AA) per dendrimer: (a) the graph of deshielding of the signals of the protons 1 to 4 and 1′ to 4′ of the dendrimer PAMAM G1 in ppm relative to N; and (b) the graph of shielding of the signals of the protons A to C of the AA in ppm relative to N.

FIG. 17 represents the graphs of the variation in the chemical shift δ (in ppm) of the signals of the protons in ¹H NMR relative to the number N of molecules of ascorbic acid (AA) per dendrimer: (a) the graph of deshielding of the signals of the protons 1 to 4 and 1′ to 2′ of the dendrimer PAMAM G4 in ppm relative to N; and (b) the graph of shielding of the signals of the protons A to C of the AA in ppm relative to N.

FIG. 18 represents the graphs of the variation in the chemical shift δ (in ppm) of the signals of the protons in ¹H NMR relative to the number N of molecules of ascorbic acid (AA) per dendrimer: (a) the graph of deshielding of the signals of the protons 1 to 3 of the dendrimer DAB G3 functionalized by the MEAC ligand in ppm relative to N; and (b) the graph of shielding of the signals of the protons A to C of the AA in ppm relative to N.

FIG. 19 represents the graphs of the variation in the chemical shift δ (in ppm) of the signals of the protons in ¹H NMR relative to the number N of molecules of ascorbic acid (AA) per dendrimer: (a) the graph of deshielding of the signals of the protons 1 to 3 of the dendrimer DAB G3 functionalized by the PFPTTEG dendron in ppm relative to N; and (b) the graph of shielding of the signals of the protons A to C of the AA in ppm relative to N.

FIG. 20 represents the graph of the variation of the chemical shift δ (or of shielding) of the signals in ¹H NMR of the protons A to C of the AA in ppm relative to the number N of molecules per AA of dendrimer of nona-amine functionalized by the PFPTTEG dendron.

### EXAMPLES

### Example 1

### Functionalization of the DAB G3 Dendrimer By the MEAC Monomer

The functionalization of the DAB G3 dendrimer by the MEAC monomer is carried out according to the following reaction, as described in the document by Kojima, C. et al. Bioconjugate Chem. (2000) 11, 910-917 (8):

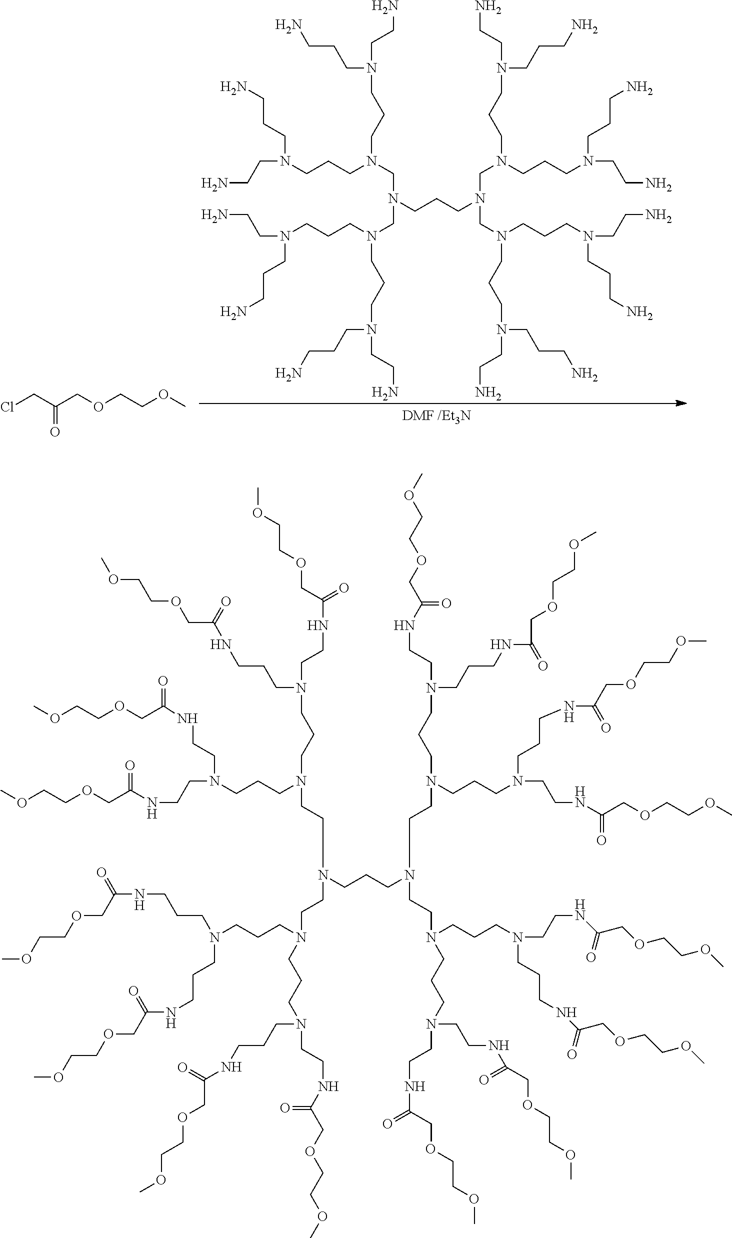

The 2,2-(methoxyethoxy)acetyl chloride dendron (210 mg: 1.38 mmol: 2 equiv. per NH₂) and also the triethylamine (185 mg: 1.84 mmol: 1.5 equiv. per NH₂) are added to a solution of DAB G3 (97 mg: 57.5 μmol) in DMF (1 ml). The mixture is stirred for 24 hours at ambient temperature, under nitrogen.

Next, 1 ml of distilled water is added to the mixture which is left stirring for 10 minutes before concentrating the product under reduced pressure. It is diluted in 5 ml of dichloromethane before extracting it into a 1% aqueous solution of potassium carbonate. The product is then purified by recrystallization in pentane, then by a chromatography column (SiO₂) with, as eluent, a chloroform/methanol mixture (95/5). 50 mg (25%) of functionalized dendrimer are obtained.

¹H NMR (CDCL₃, 250 MHz); δ ppm: 1.67 (m, CH₂—CH₂—N); 2.43 (m, CH₂—N); 3.31 (s, CH₂—NH); 3.39 (s, CH₃—O); 3.58 (m, CH₂—CH₂—O); 3.97 (m, CH₂—CO); 7.34 (s, NH—CO).

### Example 2

### Synthesis of the PFPTTEG Dendron

The synthesis of the pentafluorophenyl tris 3,4,5-tri(triethyleneoxy)benzoate dendron, described in the document by Baars, M. W. P. L. et al., Angew. Chem. Int. Ed. (2000), 39(7), 1285-1288 (9), is carried out according to the following succession of reaction steps:
Synthesis of monomethyl triethylene glycol monotosylate;
Synthesis of tris 3,4,5-tri(triethyleneoxy)benzoic acid;
Synthesis of pentafluorophenyl tris 3,4,5-tri(triethyleneoxy)benzoate.

### Synthesis of monomethyl triethylene glycol monotosylate

Monomethyl triethylene glycol monotosylate is synthesized according to the following reaction:

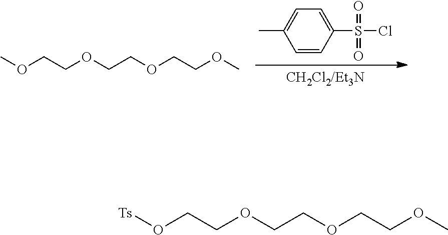

Added to a solution of monomethyl triethylene glycol (16.416 g: 99.775 mmol) in CH₂Cl₂ (10 ml), are triethylamine (199.95 mmol: 2 equiv.) and p-toluenesulfonyl chloride (TsCl: 149.96 mmol: 1.5′ equiv.). After stirring for 24 hours, the mixture is washed twice with aqueous NaHCO₃. The organic phase is dried using anhydrous sodium sulfate, filtered and evaporated under vacuum. The residue is purified in a chromatography column (SiO₂) and eluted with a petroleum ether/ethyl acetate mixture.

17.8 g (56%) of monomethyl triethylene glycol monotosylate are obtained.

¹H NMR (CDCL₃, 250 MHz); δ ppm: 2.45 (s, CH₂—Cₐᵣₒₘ); 3.37 (s, CH₃—O); 3.59 (t, CH₂—O); 4.16 (t, CH₂—O—S); 7.36 (d, CHₐᵣₒₘ—C—CH₃); 7.78 (d, CHₐᵣₒₘ—C—S).

### Synthesis of tris 3,4,5-tri(triethyleneoxy)benzoic acid

Tris 3,4,5-tri(triethyleneoxy)benzoic acid is synthesized according to the following reaction:

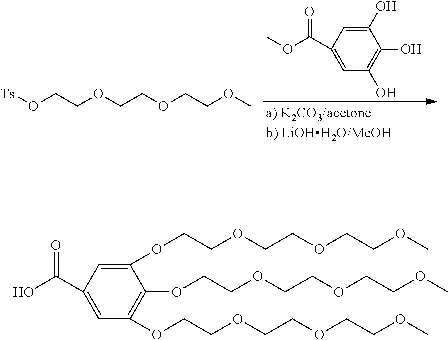

Added to a solution of trihydroxybenzoate methyl ester (0.329 g: 1.79 mmol) in acetone (10 ml) are potassium carbonate (1.772 g: 0.018 mol: 3.5 equiv.) and monomethyl triethylene glycol monotosylate (2 g: 0.006 mol: 3.5 equiv.). The heterogeneous mixture is heated at reflux for 24 hours under nitrogen; the pink mixture obtained is then filtered in order to remove excess potassium carbonate. The acetone is then evaporated and the product is dissolved in dichloromethane (12 ml). Three washings follow with 10 ml of water, then 5 ml of 1M HCl and finally 10 ml of water, before evaporating the solvent of the organic phase. The product is then purified in a chromatography column (SiO₂) with, as eluent, a 97/3 chloroform/methanol mixture.

8 g (80%) of tris 3,4,5-tri(triethyleneoxy)benzoate methyl ester are then obtained.

¹H NMR (CDCL₃, 250 MHz); δ ppm: 3.37 (s, CH₃—O); 3.65 (m, CH₂—O); 3.88 (s, CH₃—O); 4.19 (s, CH₂—O—Cₐᵣₒₘ); 7.29 (s, CHₐᵣₒₘ).

The tris 3,4,5-tri(triethyleneoxy)benzoate methyl ester (3 g: 4.823 mmol) is added to a solution of LiOH (251 mg: 6.029 mmol: 1.25 equiv.) in a water/methanol mixture (10 ml, 1/3 v/v), before being mixed for 12 hours. The mixture is evaporated under vacuum, dissolved again in water and extracted with dichloromethane in order to remove the salts.

2.4 g (80%) of 3,4,5-tri(triethyleneoxy)benzoic acid are obtained.

¹H NMR (CDCL₃, 250 MHz); δ ppm: 3.32 (s, CH₃—O); 3.61 (m, CH₂—O); 4.00 (s, CH₂—O—Cₐᵣₒₘ); 7.26 (s, CHₐᵣₒₘ)

### Synthesis of pentafluorophenyl tris 3,4,5-tri(triethyleneoxy)benzoate

Pentafluorophenyl tris 3,4,5-tri(triethyleneoxy)benzoate is synthesized according to the following reaction:

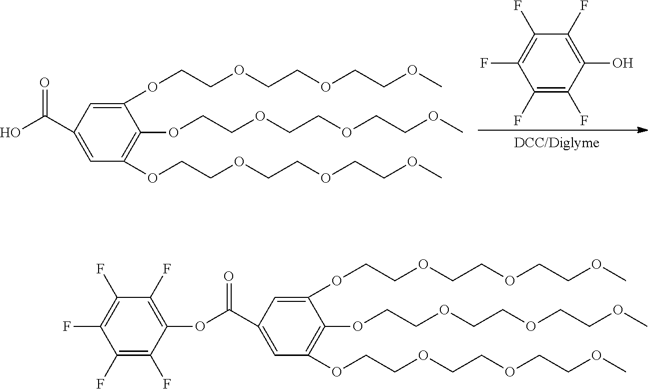

A solution of tris 3,4,5-tri(triethyleneoxy)benzoic acid (2.4 g: 3.846 mmol) and of pentafluorophenol (757 mg: 4.115 mmol: 1.07 equiv.) is prepared in diglyme (4 ml). Added to this homogeneous solution cooled to 0° C. is 1,3-dicyclocarbodiimide (DCC) (889 mg: 4.308 mmol: 1.12 equiv.). After complete addition of DCC, the reaction medium is left to return to ambient temperature. After 24 hours, the reaction mixture is filtered and this filtrate is concentrated under reduced pressure. The product is finally precipitated with cyclohexane and purified via a chromatography column (SiO₂) with, as eluent, the chloroform/methanol mixture (9/1).

1.7 g (56%) of pentafluorophenyl tris 3,4,5-tri(triethyleneoxy)benzoate are obtained.

¹H NMR (CDCL₃, 250 MHz); δ ppm: 3.32 (s, CH₃—O—CH₂); 3.61 (m, CH₂—O); 4.23 (m, CH₃—O—C); 7.44 (s, CHₐᵣₒₘ).

### Example 3

### Functionalization of the DAB G3 Dendrimer By the PFPTTEG Dendron

The functionalization of the DAB G3 dendrimer by the PFPTTEG dendron is carried out according to the following reaction:

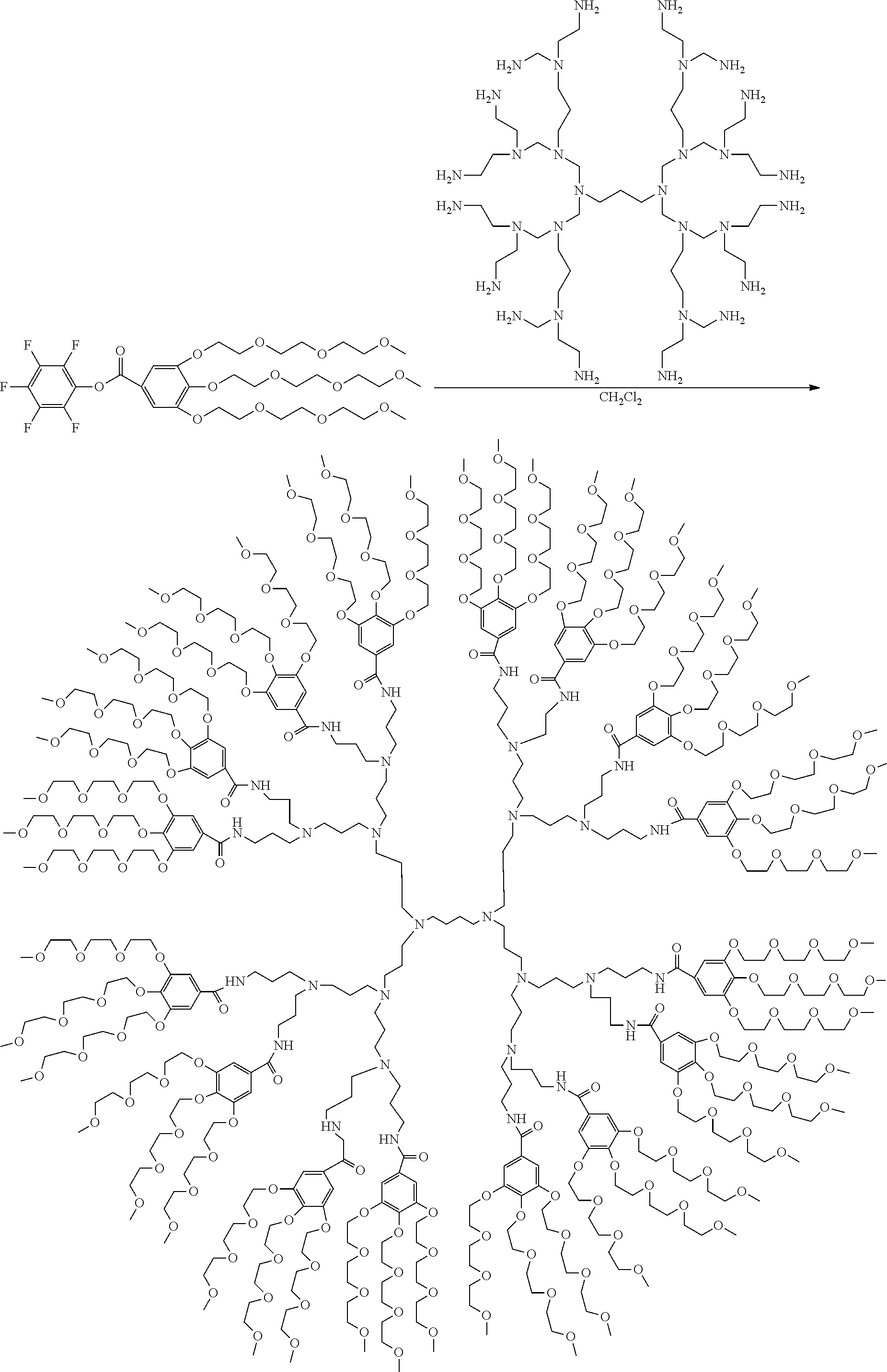

Added to a solution of DAB G3 (12 mg: 7.114 μmol) in dichloromethane (8 ml) is the pentafluorophenyl tris 3,4,5-tri(triethyleneoxy)benzoate dendron (92 mg: 116.67 μmol: 1.025 equiv. per NH₂). The mixture is stirred for 12 hours before being extracted with 0.1M NaOH. After evaporation of the aqueous phase, the product is dissolved in dichloromethane, and precipitated by addition of petroleum ether.

A few milligrams of product are obtained in a sufficient amount to carry out the titration thereof.

¹H NMR (CDCL₃, 250 MHz); δ ppm: 1.72 (m, CH₂—CH₂—NH); 2.46 (m, CH₂—N); 2.96 (m, CH₂—NH); 3.45 (s, CH₃—O); 3.65 (m, CH₂—CH₂—O); 4.15 (m, CH₂—O—Cₐᵣₒₘ); 5.43 (s, NH—CO); 7.05 (s, CHₐᵣₒₘ).

### Example 4

### Functionalization of the Nona-Amine Dendrimer By the PFPTTEG Dendron

The functionalization of the nona-amine dendrimer is carried out according to the following reaction:

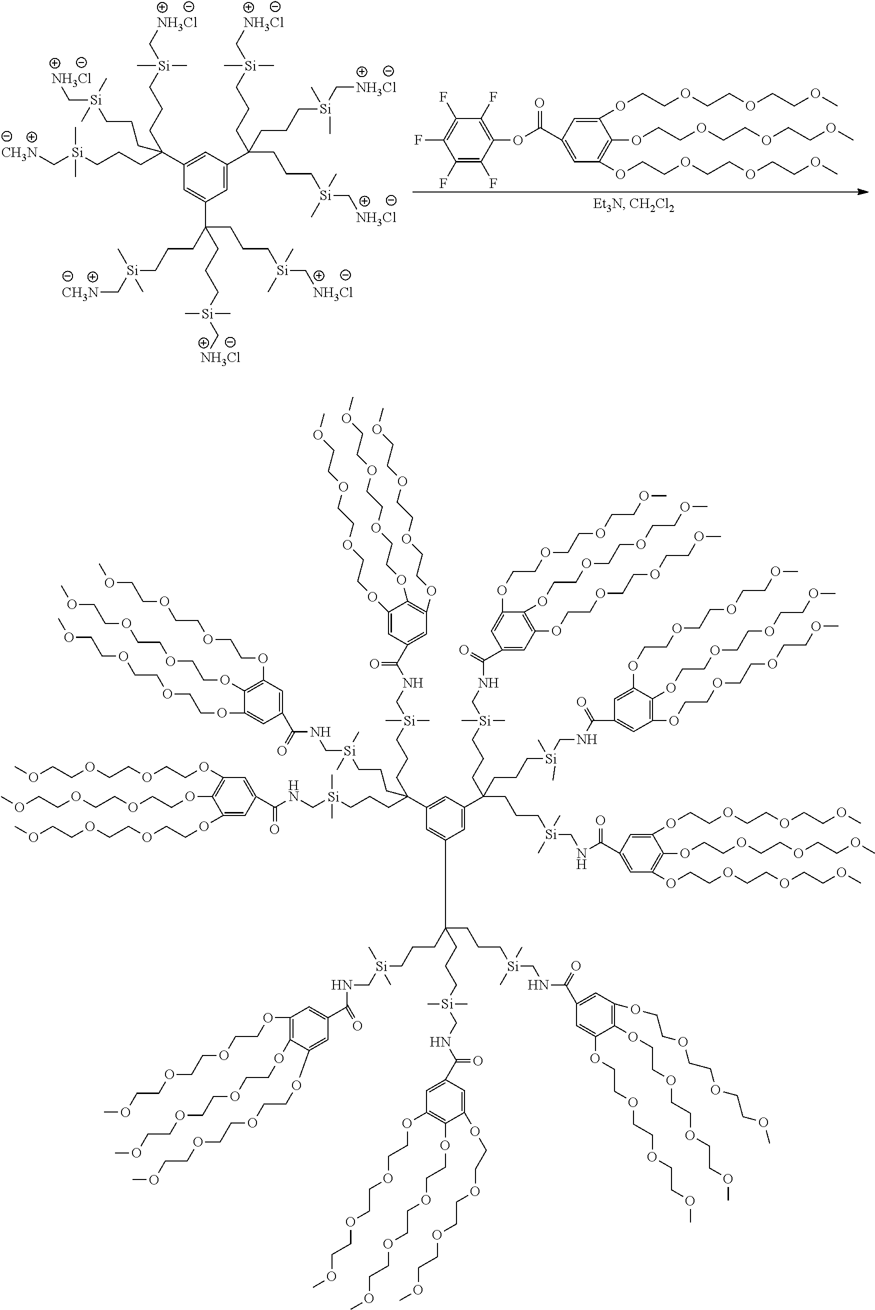

The nona-amine dendrimer (50 mg: 31 μmol) and also the PFPTTEG dendron (226 mg: 286 μmol: 1.025 equiv. per amine) are dissolved in 50 ml of distilled dichloromethane, before adding triethylamine (56 mg: 558 μmol: 2 equiv. per amine) thereto. The mixture is stirred under nitrogen at ambient temperature for 12 hours.

The mixture is dried under vacuum, then the product is again dissolved in dichloromethane before being extracted with 150 ml of a 0.1M NaOH solution. It is then washed three times with pentane, then purified by recrystallization in dichloromethane by pentane.

112 mg (55%) of nona-amine dendrimer functionalized by the PFPTTEG dendron are obtained.

¹H NMR (CDCL₃, 250 MHz); δ ppm: −0.13 (s, CH₃−Si); 0.54 (s, CH₂−Si); 1.08 (s, CH₂—CH₂—Si); 1.87 (s, CH₂—CH₂—CH₂—Si); 2.85 (s, Si—CH₂—N); 3.32 (s, CH₃—O); 3.62 (s, CH₂—CH₂—O); 4.13 (s, CH₂—O—C_{dendron}); 6.96 (s, CH_{arom dendrimer}); 7.18 (s, CH_{arom dendron}).

IR: ν=3320 cm⁻¹ and ν=1625 cm^{31 1} (NH—CO)

Elemental analysis: calculated: C 57.33; H 8.57; N 1.91; O 28.36; Si 3.83; found: C 55.07; H 8.55.

### Example 5

### Encapsulation of Vitamin C Using Some Commercial Dendrimers

Five titrations of AA were carried out: three with the

DAB dendrimers of generation 2 (denoted by DAB G2), of generation 3 (denoted by DAB G3) and of generation 5 (denoted by DAB G5) and two others with the PAMAM dendrimers of generation 1 (denoted by PAMAM G1) and of generation 4 (denoted by PAMAM G4).

The interpretation of the NMR data will be explained in detail in the case of a single generation for each dendrimer (DAB G2 and PAMAM G4); for the other titrations, a diagram of the dendrimer and also two summary graphs of the NMR data will be presented. A final table summarizing all the results obtained will end this example.

It should be noted that the following reactions are analyzed as soon as they are carried out (about ten seconds separates the two operations) which is an additional advantage for the future cosmetic use of the conjugate since its instantaneous synthesis would make it possible to optimize the formulation time.

a) DAB G2

Procedure for Encapsulating Vitamin C in DAB G2:

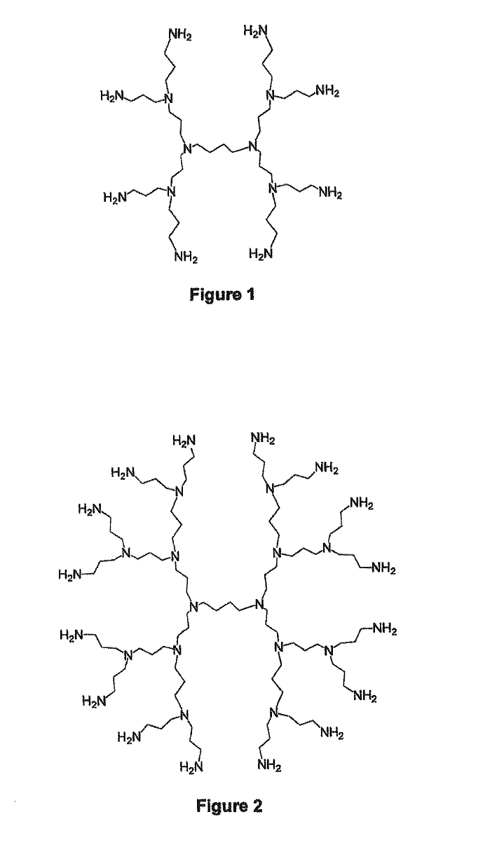

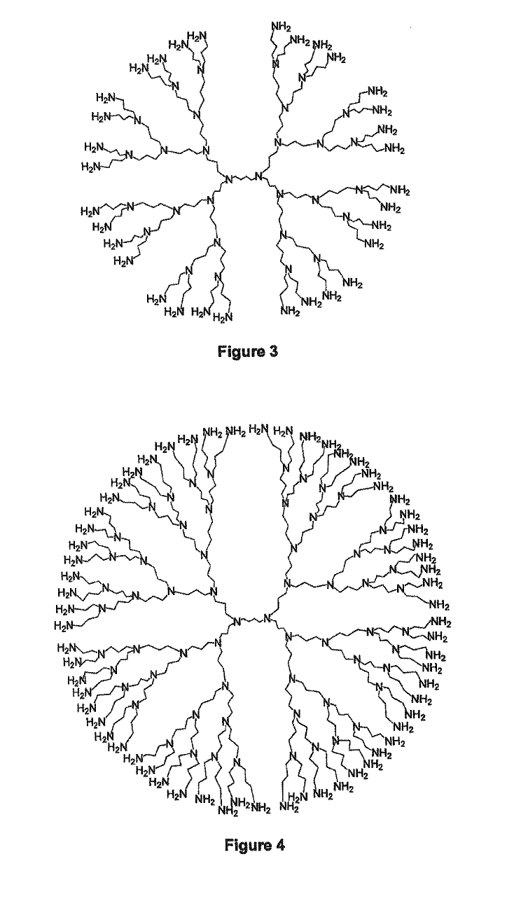

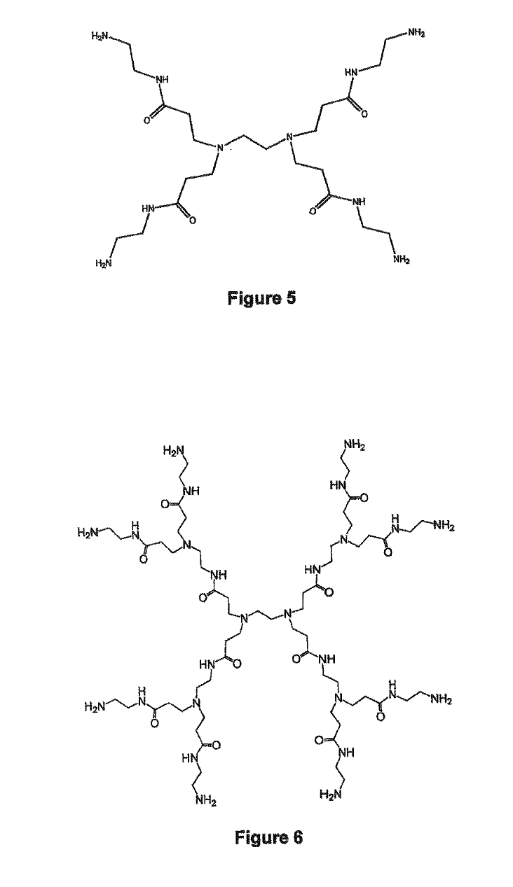

Fifteen samples E (numbered 1 to 15) having increasing concentrations of ascorbic acid (AA) relative to the DAB G2 dendrimer (FIG. 1) were prepared as follows:
A solution S1 of ascorbic acid is obtained by dissolving 170.7 mg of ascorbic acid (AA) in 1.5 ml of D₂O. The ascorbic acid used here is sold under the reference 255564 by Sigma-Aldrich (France).
Each sample E was prepared by adding a volume V of solution S1 of ascorbic acid to an NMR tube containing 5 mg of DAB G2 dendrimer in 300 μl of D₂O. This dendrimer is sold under the reference 679895 by Sigma-Aldrich (France).

The volume V introduced, the ratio of the number of molecules of AA introduced per DAB G2 dendrimer, and the mass of AA introduced into the NMR tube for each sample are listed in table 2 below.

TABLE 2

| | | | Ratio of the |
|---|---|---|---|
| | Volume V of | Mass of AA | number of |
| | solution S1 | introduced | molecules of AA |
| E | introduced (μL) | (mg) | per DAB G2 |
| 1 | 0 | 0 | 0 |
| 2 | 10 | 1.138 | 1 |
| 3 | 30 | 3.414 | 3 |
| 4 | 50 | 5.690 | 5 |
| 5 | 80 | 9.104 | 8 |
| 6 | 100 | 11.380 | 10 |
| 7 | 120 | 13.656 | 12 |
| 8 | 140 | 15.932 | 14 |
| 9 | 180 | 20.484 | 18 |
| 10 | 220 | 25.036 | 22 |
| 11 | 260 | 29.588 | 26 |
| 12 | 280 | 31.864 | 28 |
| 13 | 360 | 40.968 | 36 |
| 14 | 420 | 47.796 | 42 |
| 15 | 500 | 56.900 | 50 |

Titration of the Ascorbic Acid Contained in the DAB G2 and Results:

The preceding samples (E1 to E15) were analyzed directly after addition of the solution of ascorbic acid without additional treatment or a waiting time since the encapsulation of vitamin C in the dendrimer is instantaneous. The NMR experiments were carried out by BRUKER AC250FT spectrometer at 25° C.

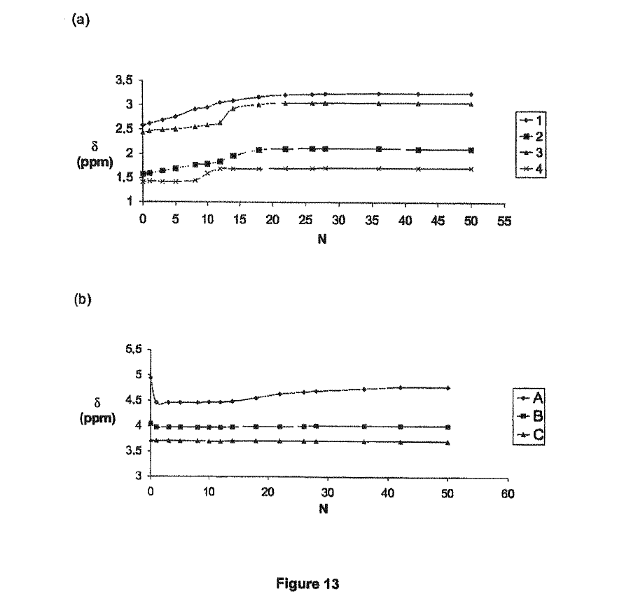

The data of the fifteen samples analyzed by NMR is given in detail in the graphs presented in FIG. 13.

The graph (a) is a representation of the various NMR data in ppm for all the hydrogens of the DAB G2 dendrimer as a function of the amount of ascorbic acid added. The protons of the dendrimer are numbered 1 to 4 according to the diagram below, in order to better visualize the shift of the NMR signals of each proton:

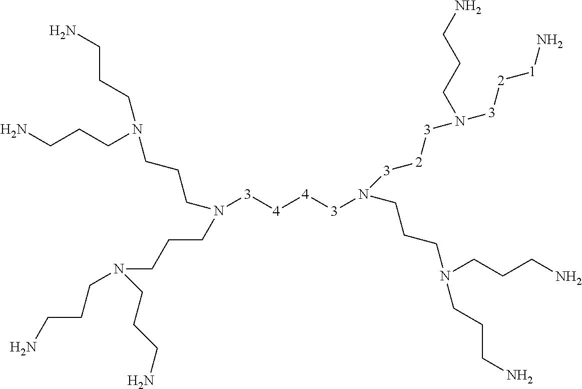

Thus, curve 1 gives the chemical shift δ in ¹H NMR of the hydrogen numbered 1 for each of the samples El to E15.

The NMR data of the peak corresponding to the protons of the primary amines are not accessible; indeed, the NMR spectra are carried out in the solvent D₂O, and these protons are in continuous exchange with the solvent hence the absence of their signals.

The signals of DAB G2 are progressively deshielded up to a concentration of around 30 AA/DAB, which means that all the primary and tertiary amines are quaternized with the AA, and a few additional AA are attached to the dendrimer securely by hydrogen bonds with the protons of the primary amines at the periphery of the dendrimer. Furthermore, the AAs are attached firstly at the periphery since the curves 1 and 2 have a greater initial slope than those of curves 3 and 4.

The graph (b) represents the various NMR data in ppm of the hydrogens of the ascorbic acid (AA) indexed from A to C as in the diagram below:

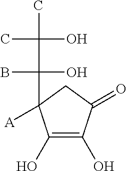

From 14 AA/DAB, the peaks of the AA begin to be shielded, which corresponds to the appearance of ascorbic acid in the solution. Therefore, an ascorbic acid/ascorbate mixture is present.

However, the curve A, which is the most significant, continues to increase up to a concentration that reaches about 60 AA/DAB. There would therefore be an AA/ascorbate equilibrium when the number of AA added lies between 14 and 60. Then, the curve plateaus, which means that beyond 60 AA added a dynamic equilibrium could be present. Furthermore, it is also possible to calculate the number of molecules of ascorbic acid attached to the dendrimer by virtue of the NMR data since, when the peak of the proton A of the ascorbic acid lies halfway between the values of this same proton in the ascorbic and ascorbate form (i.e. δ=4.7 ppm), then it can be considered that half the molecules of ascorbic acid capable of being transported are attached. This dendrimer therefore should make it possible to transport up to 60 AA.

It should be noted that a titration of propionic acid was also carried out with the DAB G2 dendrimer in order to endorse the existence of encapsulation and of hydrogen bonds between the AA and the dendrimer. Indeed, the results obtained show the attachment of only eight propionic acids, which corresponds to the formation of eight ammonium propionates at the periphery of the dendrimer.

The alcohol functional groups present on the ascorbic acid would then form hydrogen bonds with the amines of the dendrimer and would also enable them to be encapsulated at its core.

b) DAB G3

Procedure for Encapsulating Vitamin C in DAB G3:

Twelve samples (E1 to E12) having increasing concentrations of ascorbic acid (AA) relative to the DAB G3 dendrimer (FIG. 2) were prepared as follows:
A solution S2 of ascorbic acid is obtained by dissolving 104.5 mg of ascorbic acid (AA) in 1 ml of D₂O.
Each sample E was prepared by adding a volume V of solution S2 of ascorbic acid to an NMR tube containing 10 mg of DAB G3 dendrimer in 300 μl of D₂O. This dendrimer is sold under the reference 469076 by Sigma-Aldrich (France).

The amounts of AA introduced and the ratio of the number of molecules of AA introduced per DAB G3 dendrimer into the NMR tube for each sample are listed in table 3 below.

TABLE 3

| | | | Ratio of the |
|---|---|---|---|
| | Volume V of | Mass of AA | number of |
| | solution S2 | introduced | molecules of AA |
| E | introduced (μL) | (mg) | per DAB G3 |
| 1 | 0 | 0 | 0 |
| 2 | 20 | 2.09 | 2 |
| 3 | 50 | 5.22 | 5 |
| 4 | 100 | 10.43 | 10 |
| 5 | 160 | 16.69 | 16 |
| 6 | 200 | 20.87 | 20 |
| 7 | 250 | 26.09 | 25 |
| 8 | 300 | 31.30 | 30 |
| 9 | 450 | 46.95 | 45 |
| 10 | 600 | 62.60 | 60 |
| 11 | 750 | 78.26 | 75 |
| 12 | 900 | 93.91 | 90 |

Titration of the Ascorbic Acid Contained in the DAB G3 and Results:

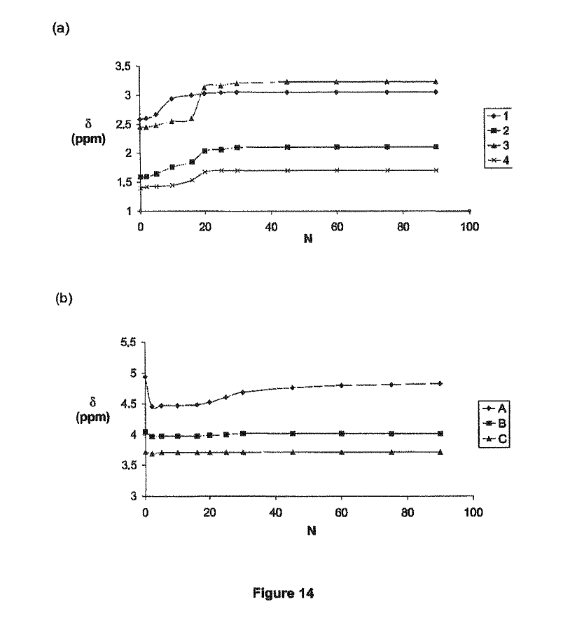

The twelve samples were then analyzed directly by NMR. The NMR data is given in detail in the graphs presented in FIG. 14.

The graph (a) represents the deshielding of the hydrogens of the dendrimer numbered from 1 to 4 as below:

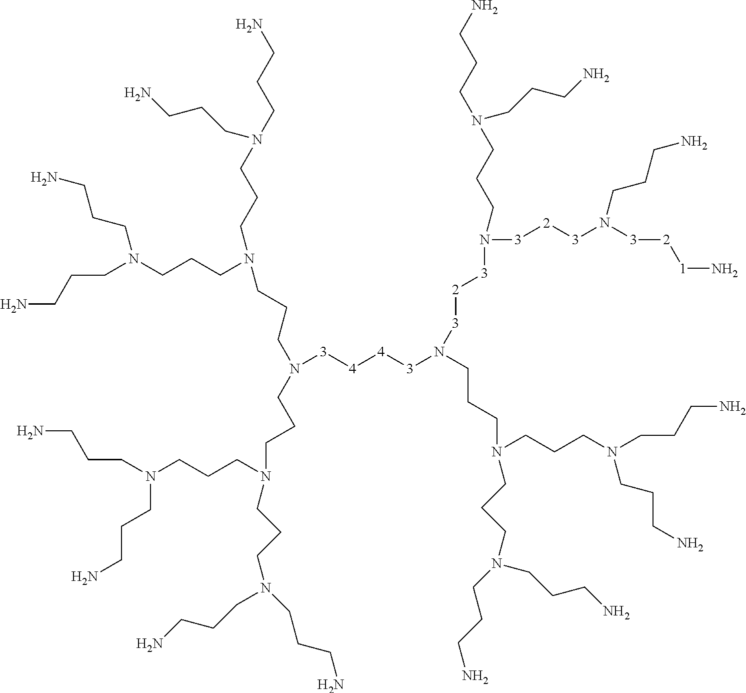

The graph (b) represents the various NMR data in ppm of the hydrogens of ascorbic acid (AA) indexed by A to C as below:

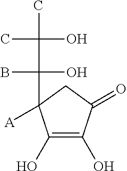

A reasoning similar to that of the preceding point a) makes it possible to conclude that, for the DAB G3 dendrimer, 16 molecules of ascorbate may be attached before the appearance of ascorbic acid and that the DAB G3 dendrimer may comprise 80 attached molecules of ascorbate.

c) DAB G5

Procedure for Encapsulating Vitamin C in DAB G5:

Fifteen samples (El to E15) comprising increasing amounts of ascorbic acid (AA) relative to the DAB G5 dendrimer (FIG. 4) were prepared as follows:
A solution S3 of ascorbic acid is obtained by dissolving 91.5 mg of ascorbic acid (AA) in 1.5 ml of D₂O.
Each sample E was prepared by adding a volume V of solution S3 of ascorbic acid to an NMR tube containing 5 mg of DAB G5 dendrimer in 300 μL of D₂O. This dendrimer is sold under the reference 469092 by Sigma-Aldrich (France).

The amounts of AA introduced and the ratio of the number of molecules of AA introduced per DAB G5 dendrimer into the NMR tube for each sample are listed in table 4 below.

TABLE 4

| | | | Ratio of the |
|---|---|---|---|
| | Volume V of | Mass of AA | number of |
| | solution S3 | introduced | molecules of AA |
| E | introduced (μL) | (mg) | per DAB G5 |
| 1 | 0 | 0 | 0 |
| 2 | 20 | 1.22 | 10 |
| 3 | 40 | 2.45 | 20 |
| 4 | 60 | 3.68 | 30 |
| 5 | 80 | 4.91 | 40 |
| 6 | 100 | 6.14 | 50 |
| 7 | 128 | 7.86 | 64 |
| 8 | 200 | 12.28 | 100 |
| 9 | 252 | 15.47 | 126 |
| 10 | 300 | 18.42 | 150 |
| 11 | 400 | 24.55 | 200 |
| 12 | 436 | 26.76 | 218 |
| 13 | 600 | 36.83 | 300 |
| 14 | 800 | 49.11 | 400 |
| 15 | 1200 | 73.66 | 600 |

Titration of the Ascorbic Acid Contained in the DAB G5 and Results:

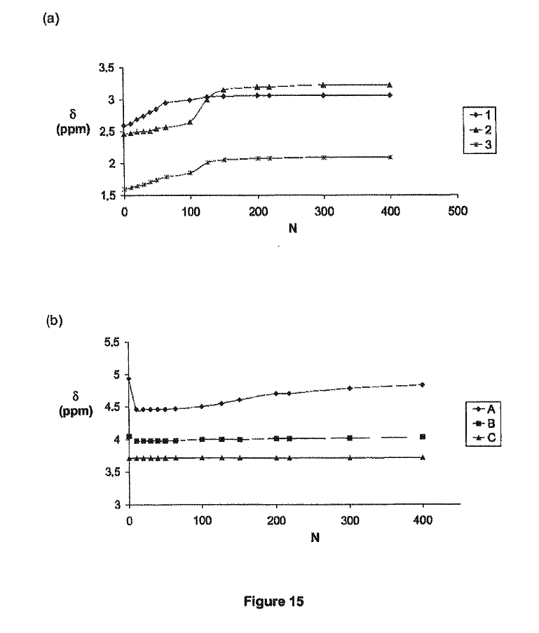

The fifteen samples were analyzed by NMR. The NMR data is given in detail in the graphs presented in FIG. 15.

The graph (a) represents the deshielding of the hydrogens of the dendrimer numbered from 1 to 4 as below:

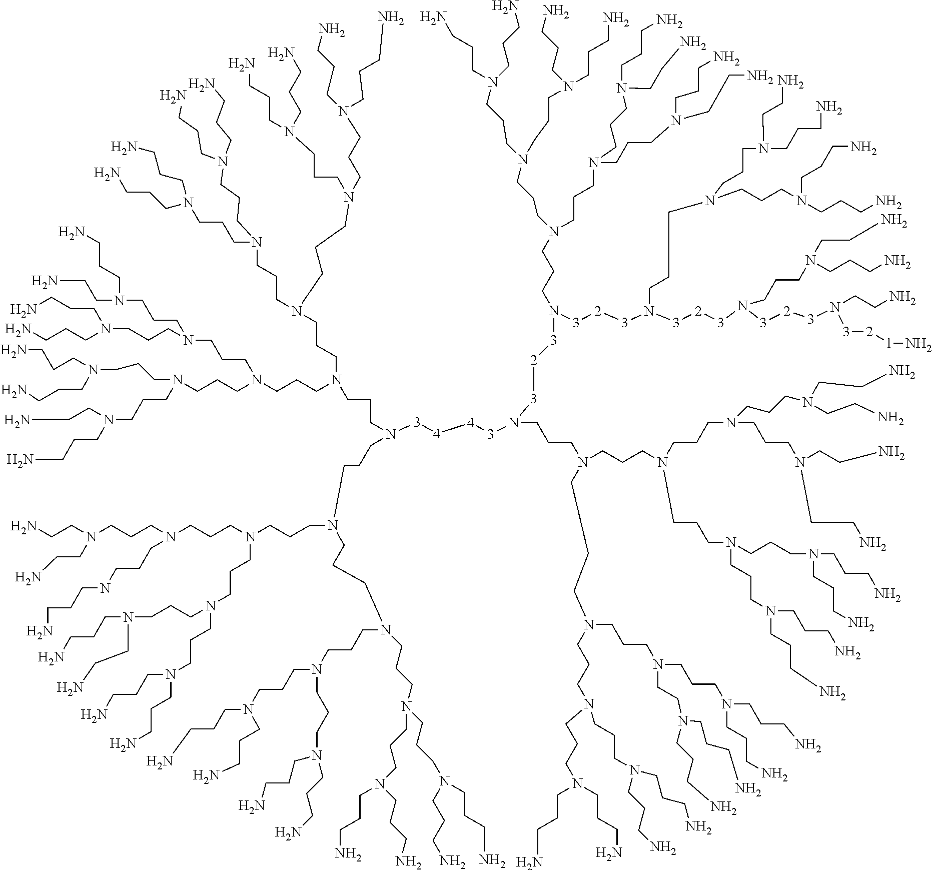

The graph (b) represents the various NMR data in ppm of the hydrogens of the ascorbic acid (AA) indexed from A to C previously.

d) PAMAM G1 Dendrimer

Procedure for Encapsulating Vitamin C in PAMAM G1:

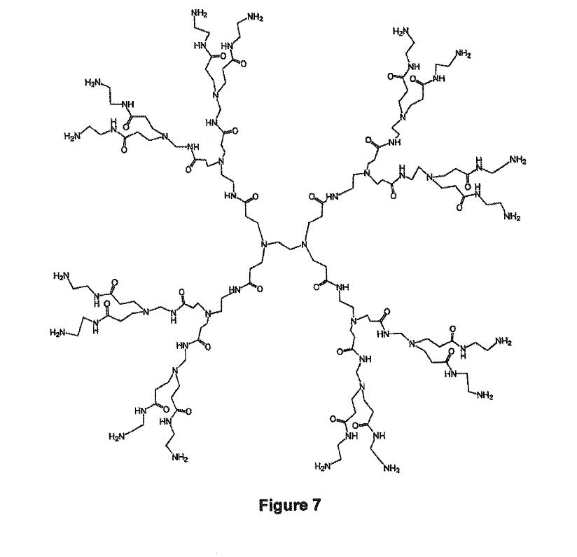

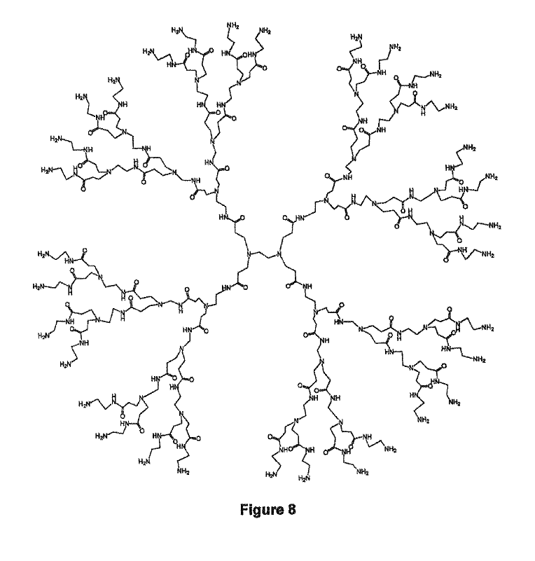

Fifteen samples (E1 to E15) comprising increasing amounts of ascorbic acid relative to the PAMAM G1 dendrimer (FIG. 6) were prepared as follows:
A solution S4 of ascorbic acid is obtained by dissolving 61.5 mg of ascorbic acid (AA) in 1 ml of D₂O.
Each sample E was prepared by adding a volume V of solution S4 of ascorbic acid to an NMR tube containing 5 mg of PAMAM G1 dendrimer in 300 μL of D₂O. This dendrimer is sold under the reference 597414 by Sigma-Aldrich (France).

The amounts of AA introduced and the ratio of the number of molecules of AA introduced per PAMAM G1 dendrimer into the NMR tube for each sample are listed in table 5 below.

TABLE 5

| | | | Ratio of the |
|---|---|---|---|
| | Volume V of | Mass of AA | number of |
| | solution S4 | introduced | molecules of AA |
| E | introduced (μL) | (mg) | per PAMAM G1 |
| 1 | 0 | 0 | 0 |
| 2 | 10 | 0.615 | 1 |
| 3 | 30 | 1.23 | 3 |
| 4 | 50 | 3.08 | 5 |
| 5 | 80 | 4.92 | 8 |
| 6 | 100 | 6.15 | 10 |
| 7 | 120 | 7.39 | 12 |
| 8 | 140 | 8.62 | 14 |
| 9 | 180 | 11.08 | 18 |
| 10 | 220 | 13.54 | 22 |
| 11 | 260 | 16.00 | 26 |
| 12 | 280 | 17.23 | 28 |
| 13 | 360 | 22.16 | 36 |
| 14 | 420 | 25.85 | 42 |
| 15 | 500 | 30.77 | 50 |

Titration of the Ascorbic Acid Contained in the PAMAM G1 and Results:

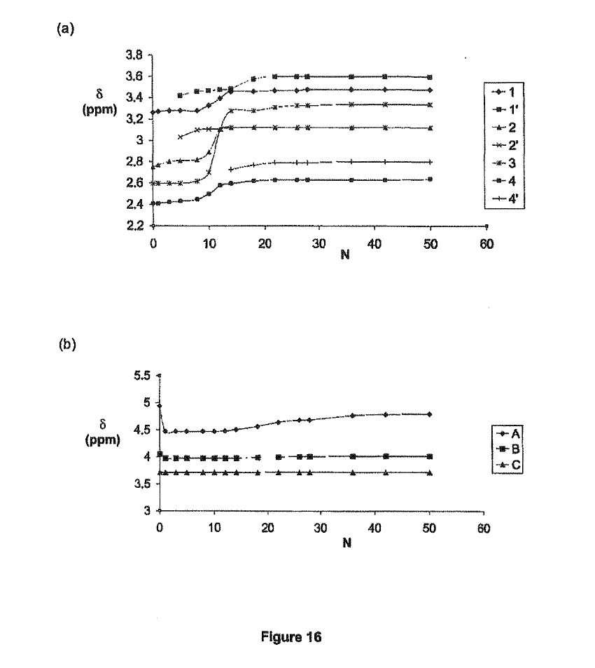

The fifteen samples were analyzed by NMR. The NMR data is given in detail in the graphs presented in FIG. 16.

The graph (a) represents the deshielding of the hydrogens of the dendrimer numbered from 1 to 4 as below:

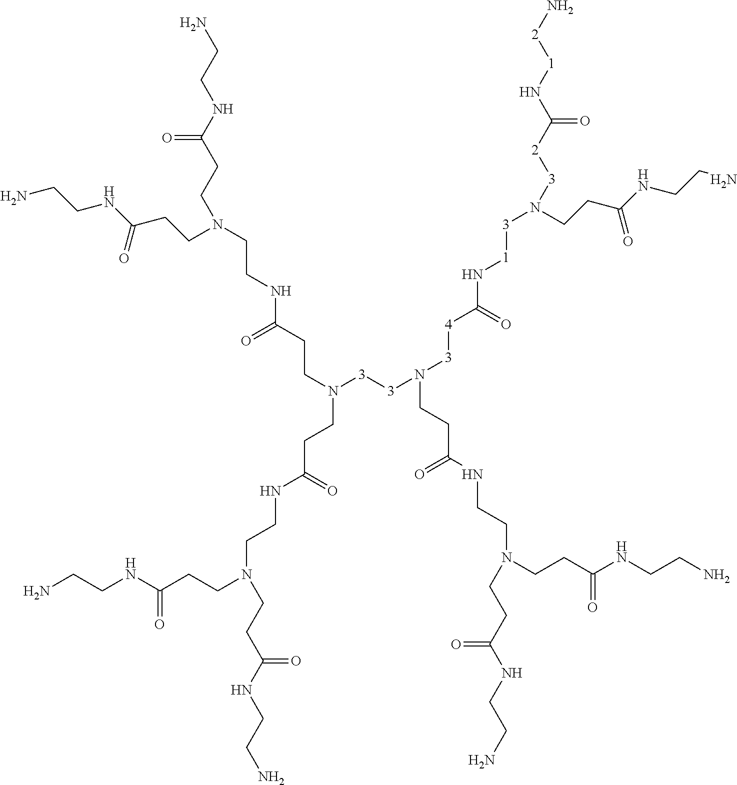

The graph (b) represents the various NMR data in ppm of the hydrogens of the ascorbic acid (AA) indexed from A to C previously.

e) PAMAM G4 Dendrimer

Procedure for Encapsulating Vitamin C in PAMAM G4:

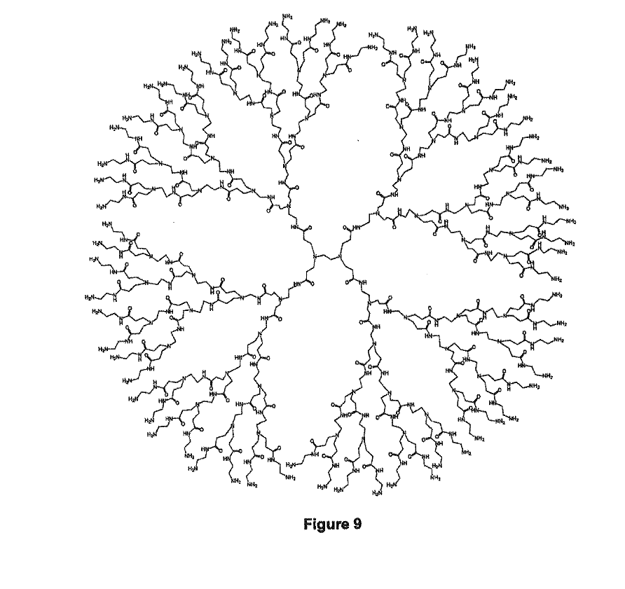

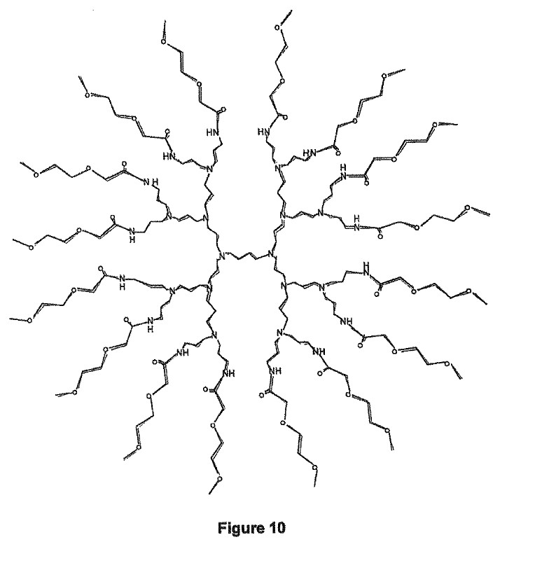

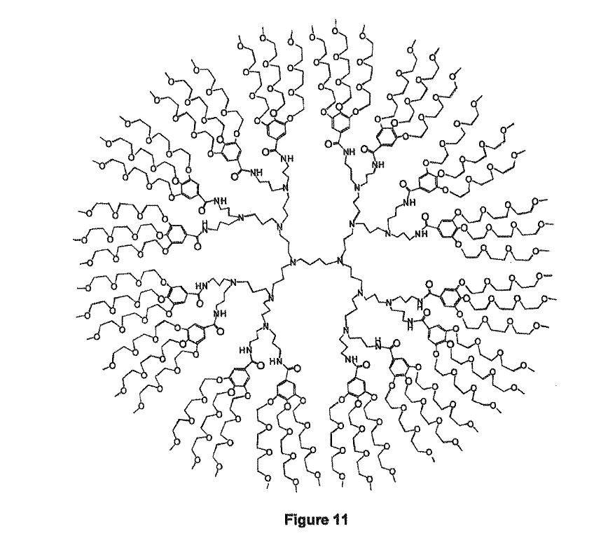

Fifteen samples (E1 to E15) comprising increasing amounts of ascorbic acid relative to the PAMAM G4 dendrimer (FIG. 9) were prepared as follows:
A solution S5 of ascorbic acid is obtained by dissolving 62 mg of ascorbic acid (AA) in 2 ml of D₂O.
Each sample E was prepared by adding a volume V of solution S5 of ascorbic acid to an NMR tube containing 5 mg of PAMAM G4 dendrimer in 300 μL of D₂O. This dendrimer is sold under the reference 597856 by Sigma-Aldrich (France).

The amounts of AA introduced and the ratio of the number of molecules of AA introduced per PAMAM G4 dendrimer into the NMR tube for each sample are listed in table 6 below.

TABLE 6

| | | | Ratio of the |
|---|---|---|---|
| | Volume V of | Mass of AA | number of |
| | solution S5 | introduced | molecules of AA |
| E | introduced (μL) | (mg) | per PAMAM G4 |
| 1 | 0 | 0 | 0 |
| 2 | 20 | 0.62 | 10 |
| 3 | 40 | 1.24 | 20 |
| 4 | 60 | 1.86 | 30 |
| 5 | 80 | 2.48 | 40 |
| 6 | 100 | 3.10 | 50 |
| 7 | 128 | 3.96 | 64 |
| 8 | 200 | 6.19 | 100 |
| 9 | 252 | 7.80 | 126 |
| 10 | 300 | 9.29 | 150 |
| 11 | 400 | 12.38 | 200 |
| 12 | 436 | 13.50 | 218 |
| 13 | 600 | 18.57 | 300 |
| 14 | 800 | 24.76 | 400 |
| 15 | 1200 | 37.14 | 600 |

Titration of the Ascorbic Acid Contained in the PAMAM G4 and Results:

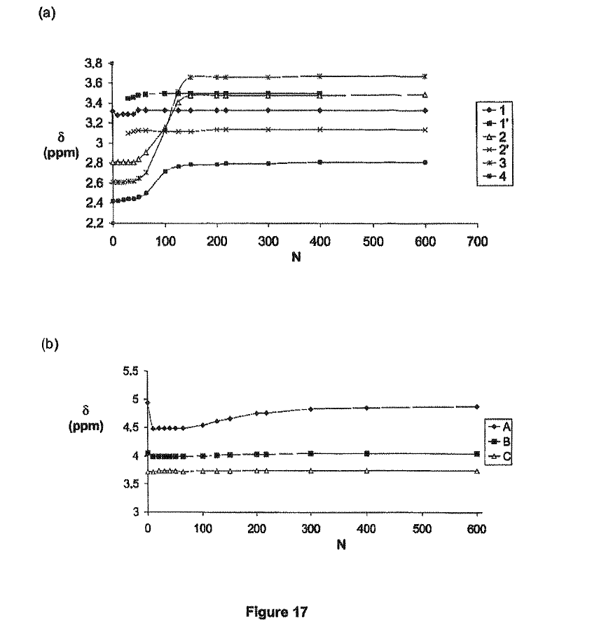

The fifteen samples were analyzed by NMR. The NMR data is given in detail in the graphs presented in FIG. 17.

The graph (a) represents the deshielding of the hydrogens of the dendrimer numbered from 1 to 4 as below:

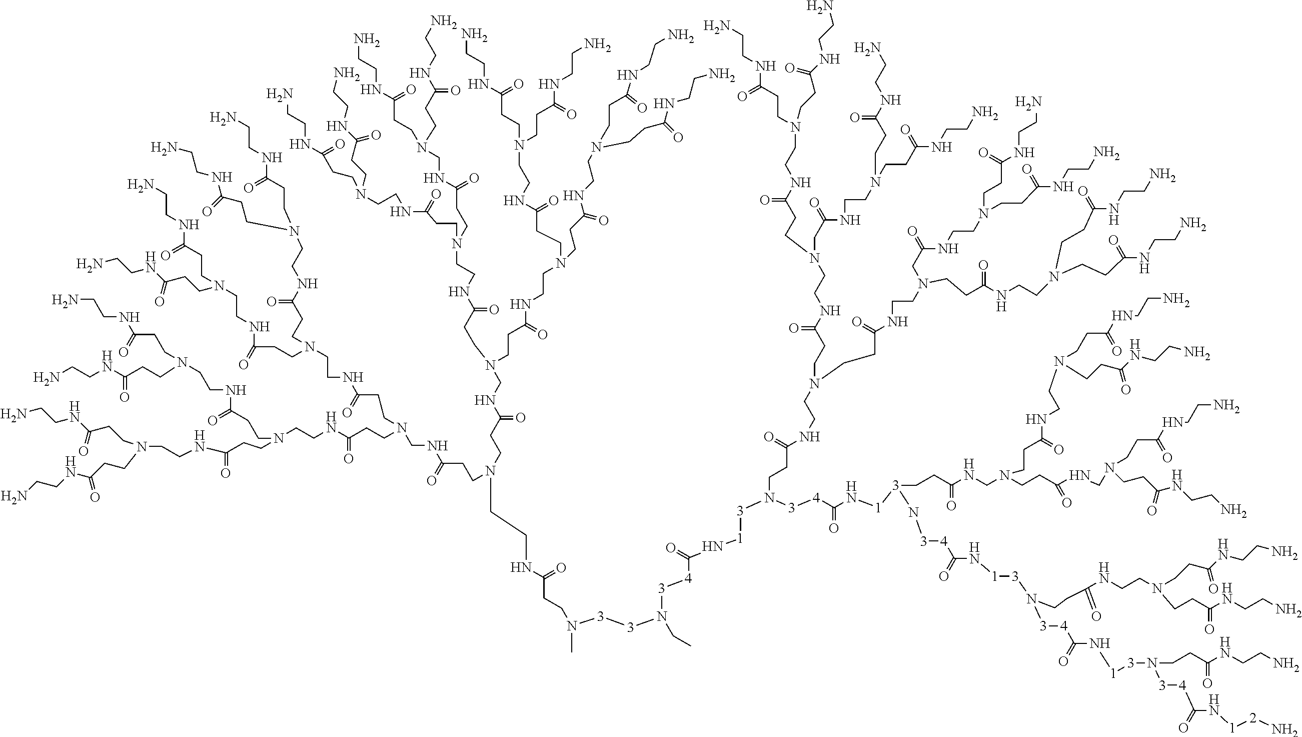

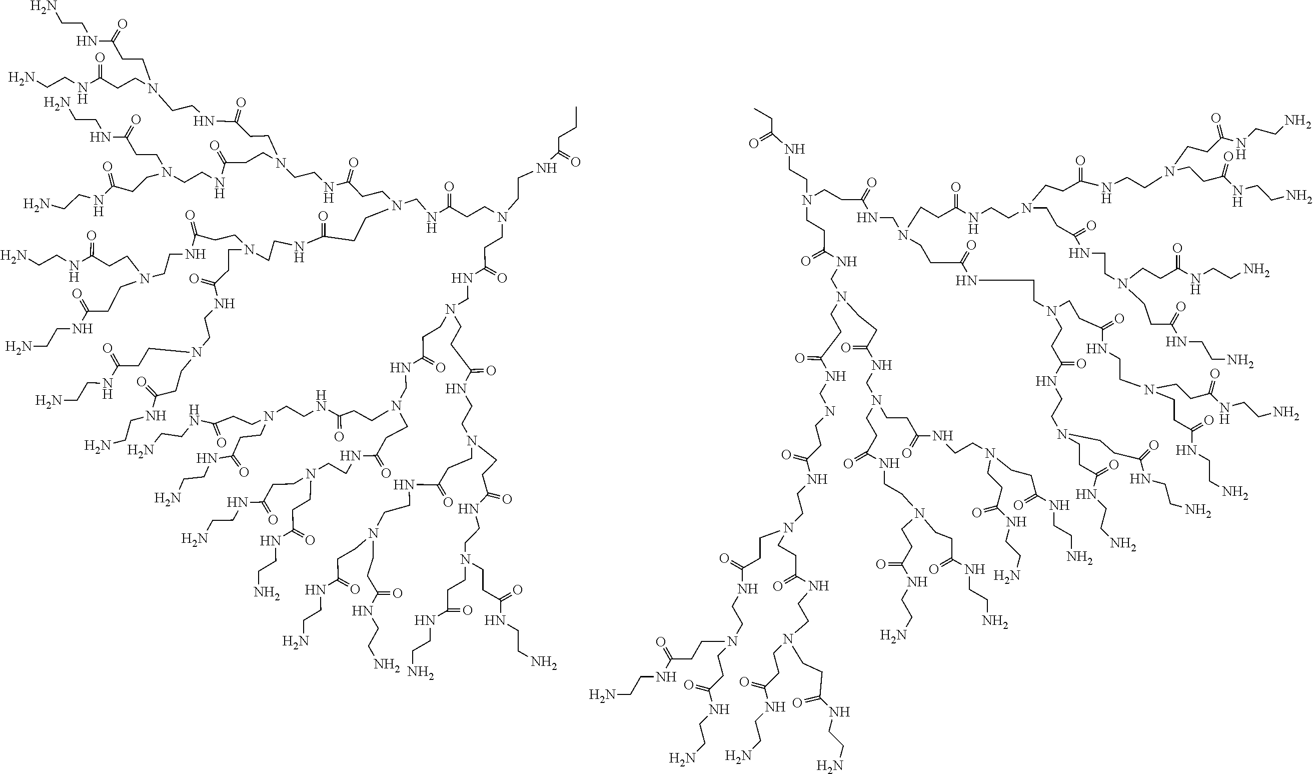

The graph (b) represents the various NMR data in ppm of the hydrogens of the ascorbic acid (AA) indexed from A to C previously.

The signals of PAMAM G4 are progressively deshielded up to a concentration of 64 AA/PAMAM. It may be considered that the AA is at the start encapsulated in the dendrimer, which generates only weak deshielding. Next, the signals 2 and 3 corresponding to the external primary amines and to the internal tertiary amines are greatly deshielded, hence the presence of ammonium ascorbate at the core and also at the periphery of the dendrimer.

This graph does not make it possible to envisage preferably as regards the attachment of the AAs to the primary or tertiary amines since the curves develop similarly. It may also be that the AA is attached progressively from generation to generation, the steric hindrance then impeding its advance toward the core of the dendrimer.

The attachment of AA by the dendrimer stagnates and the curves then form a plateau when the concentration reaches 400 AA/PAMAM; this implies that most of the amines of the PAMAM dendrimer are quaternized and that some additional AAs are attached to the dendrimer either by encapsulation in its pores, or by hydrogen bonds with the protons of the primary amines at its periphery.

Up to a concentration of 64 AA/PAMAM, the peaks of the AA are deshielded, then they begin to be shielded, which could suggest a first attachment of the AAs to the 64 external primary amines.

Next several hundreds of ascorbic acid molecules appear to be attached to the core of the dendrimer and also at the periphery by hydrogen bonds, while others remain in the acid form in solution.

To conclude, the fact that the peaks 1 and 2 split up respectively into 1′ and 2′ may be explained by the number of generations of the dendrimer. Indeed, the amines react differently with the AA depending on the generation to which they belong, which is explained by a hindered access according to the depth of the amine at the core of the dendrimer.

f) Conclusion

Table 7 below summarizes the results of all the preceding titrations by giving the number of ammonium ascorbates formed before and after appearance of ascorbic acid in the medium:

TABLE 7

| | Number of ascorbate | Total number of |
|---|---|---|
| | molecules attached | ascorbate |
| | before appearance | molecules |
| Dendrimer | of ascorbic acid | attached |
| DAB G2 | 14 | 60 |
| DAB G3 | 16 | 80 |
| DAB G5 | 64 | 400 |
| PAMAM G1 | 14 | 60 |
| PAMAM G4 | 64 | 400 |

The results obtained are consistent with those expected; indeed, the higher the generation of the dendrimer, the higher the number of molecules of ascorbic acid transported. Furthermore, it should be noted that the nature of the dendrimer (DAB or PAMAM) has no influence on the attachment since the results are identical in both cases; only the number of primary and tertiary amines present at the periphery and at the core of the dendrimer matter (it is recalled that the dendrimers DAB G2 and PAMAM G1, and also DAB G5 and PAMAM G4 have an equivalent number of amine functional groups).

The desired objective, which was to visualize the existence of bonds between vitamin C and the dendrimers, is therefore achieved. It now remains to carry out the same type of experiments and also additional experiments in order to optimize the result obtained and to find a good compromise between transport efficiency, solubility and toxicity.

### Example 6

### Encapsulation of Vitamin C Using Some Functionalized Dendrimers

The various advantages provided by a graft of polyethylene glycol chains at the periphery of a molecule have already been demonstrated many times. Specifically, this water-soluble, non-toxic and non-immunogenic polymer is highly suitable for biological use owing to a high water solubility and biocompatibility capacity.

It was therefore judicious to functionalize the dendrimers with two different molecules containing either one (MEAC monomer) or three PEG chains (PFPTTEG dendron) in order to compare the properties provided by each of them.

a) The DAB G3 Dendrimer Functionalized by the MEAC Monomer

The functionalization of the DAB G3 dendrimer with the MEAC monomer is carried out according to the synthesis described in example 1.

Procedure for Encapsulating Vitamin C:

Fourteen samples (E1 to E14) comprising increasing amounts of ascorbic acid compared to the DAB G3 dendrimer, functionalized by the MEAC monomer (FIG. 10) were prepared as follows:
A solution S6 of ascorbic acid is obtained by dissolving 49 mg of ascorbic acid (AA) in 2 ml of D₂O.
Each sample E was prepared by adding a volume V of solution S6 of ascorbic acid into an NMR tube containing 5 mg of DAB G3 dendrimer functionalized by the MEAC monomer in 300 μl of D₂O.

The amounts of AA introduced and the ratio of the number of molecules of AA introduced per dendrimer into the NMR tube for each sample are listed in table 8 below.

TABLE 8

| | | | Ratio of the |
|---|---|---|---|
| | | | number of |
| | | | molecules of AA |
| | Volume V of | Mass of AA | per DAB G3 |
| | solution S6 | introduced | functionalized by |
| E | introduced (μL) | (mg) | the MEAC monomer |
| 1 | 0 | 0 | 0 |
| 2 | 20 | 0.497 | 2 |
| 3 | 50 | 1.243 | 5 |
| 4 | 100 | 2.486 | 10 |
| 5 | 160 | 3.977 | 16 |
| 6 | 200 | 4.972 | 20 |
| 7 | 250 | 6.215 | 25 |
| 8 | 300 | 7.458 | 30 |
| 9 | 450 | 11.186 | 45 |
| 10 | 600 | 14.915 | 60 |
| 11 | 750 | 18.644 | 75 |
| 12 | 900 | 22.373 | 90 |
| 13 | 1000 | 24.859 | 100 |
| 14 | 1500 | 37.288 | 150 |

Titration of the Ascorbic Acid Contained in the Dendrimer and Results:

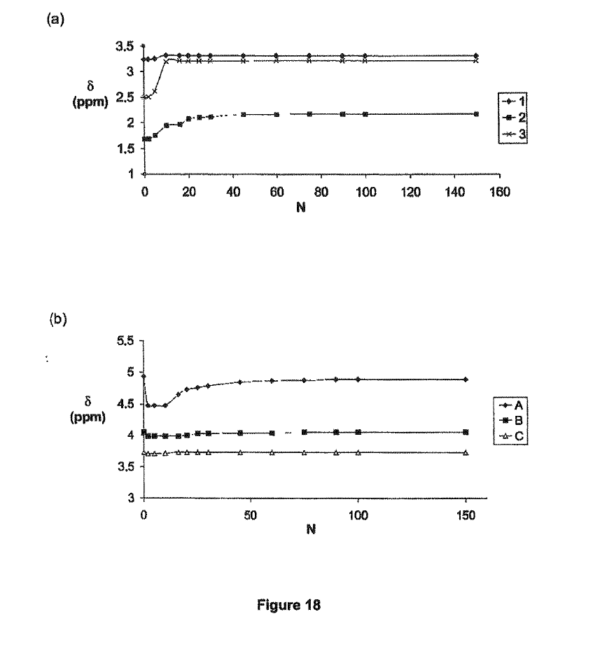

The NMR titration of the ascorbic acid with this functionalized dendrimer is carried out in order to compare its vitamin C transport properties. The NMR data is given in detail in the graphs presented in FIG. 18.

The graph (a) represents the deshielding of the hydrogens of the dendrimer numbered from 1 to 4 as below:

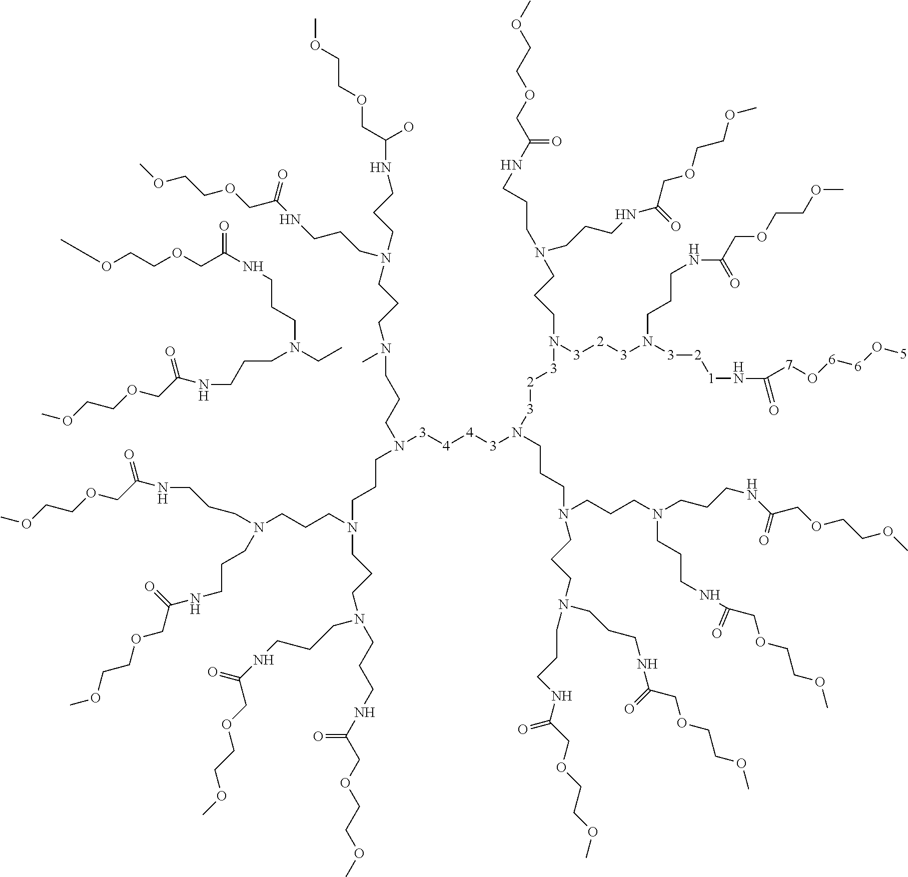

The graph (b) represents the various NMR data in ppm of the hydrogens of ascorbic acid (AA) indexed from A to C previously.

According to the results obtained, it is clear that this dendrimer transports vitamin C with a similar efficiency to the non-functionalized DAB G3 dendrimer but with the advantages of much better solubility and biocompatibility. Indeed, this dendrimer may attach 16 molecules of ascorbate before appearance of ascorbic acid molecules, and up to 80 molecules of ascorbate before reaching a dynamic equilibrium. As regards the NMR data of the dendron (5, 6 and 7), they do not vary at all during the titration; this means that the monoethylene glycolated branches do not participate in the attachment of the AAs, nor in an encapsulation within the dendrimer.

b) The DAB G3 Dendrimer Functionalized by the PFPTTEG Dendron

The PFPTTEG dendron is first synthesized according to example 2 in order to carry out the functionalization of the DAB G3 dendrimer according to the procedure described in example 3.

Procedure for Encapsulating Vitamin C:

Fourteen samples (E1 to E14) comprising increasing amounts of ascorbic acid compared to the DAB G3 dendrimer, functionalized by the PFPTTEG dendron (FIG. 11) were prepared as follows:
A solution S7 of ascorbic acid is obtained by dissolving 15.8 mg of ascorbic acid (AA) in 2 ml of D₂O.
Each sample E was prepared by adding a volume V of solution S7 of ascorbic acid into an NMR tube containing 5 mg of DAB G3 dendrimer functionalized by the PFPTTEG dendron in 300 μl of D₂O.

The amounts of AA introduced and the ratio of the number of molecules of AA introduced per dendrimer into the NMR tube for each sample are listed in table 9 below.

TABLE 9

| | | | Ratio of the |
|---|---|---|---|
| | | | number of |
| | | | molecules of AA |
| | | | per DAB G3 |
| | Volume V of | Mass of AA | functionalized by |
| | solution S7 | introduced | the PFPTTEG |
| E | introduced (μL) | (mg) | dendron |
| 1 | 0 | 0 | 0 |
| 2 | 20 | 0.158 | 2 |
| 3 | 50 | 0.396 | 5 |
| 4 | 100 | 0.791 | 10 |
| 5 | 160 | 1.266 | 16 |
| 6 | 200 | 1.582 | 20 |
| 7 | 250 | 1.978 | 25 |
| 8 | 300 | 2.373 | 30 |
| 9 | 450 | 3.559 | 45 |
| 10 | 600 | 4.746 | 60 |
| 11 | 750 | 5.933 | 75 |
| 12 | 900 | 7.119 | 90 |
| 13 | 1000 | 7.911 | 100 |
| 14 | 1500 | 11.865 | 150 |

Titration of the Ascorbic Acid Contained in the Dendrimer

The NMR titration of AA is carried out in order to evaluate the transport capacity of vitamin C by the DAB G3 dendrimer functionalized with the PFPTTEG dendron.

The dendrimer obtained is soluble in a wide range of solvents, from the most apolar such as toluene and dichloromethane to the most polar such as water, methanol and acetonitrile. Furthermore, this functionalized dendrimer is soluble in water in all proportions, which is an additional advantage allowing a high efficiency of transported AA.

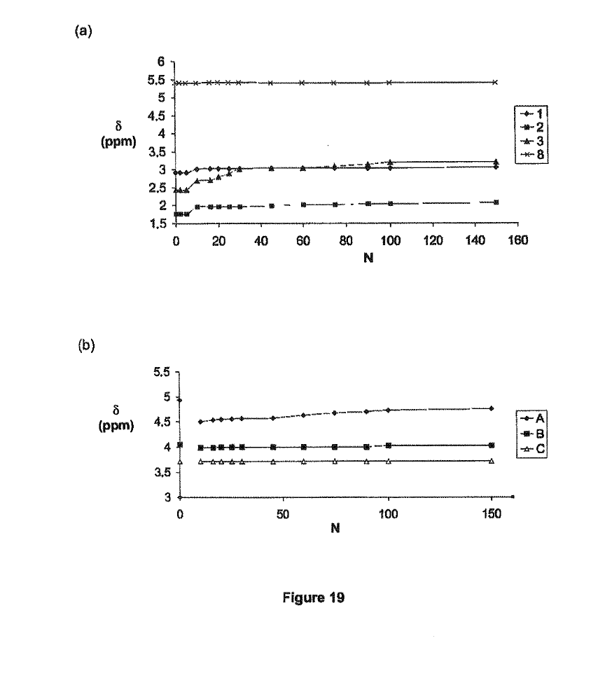

The NMR titration of ascorbic acid with this functionalized dendrimer is carried out in order to compare its vitamin C transport properties. The NMR data are given in detail in the graphs presented in FIG. 19.

The graph (a) represents the deshielding of the hydrogens of the dendrimer numbered from 1 to 4 as below:

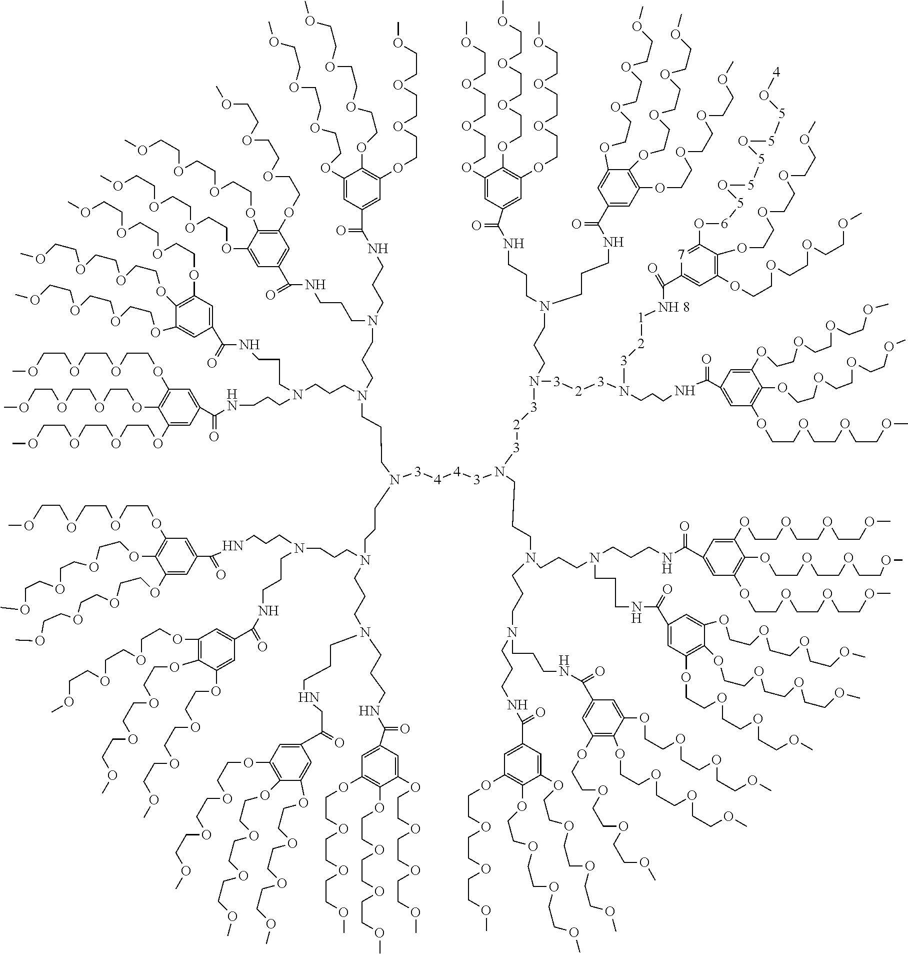

According to the results obtained, it is obvious that the functionalization of the dendrimer would not in any case disrupt the transport of vitamin C, quite the contrary. Indeed, this dendrimer can attach 45 molecules of ascorbate before appearance of molecules of ascorbic acid, and up to 100 molecules of ascorbate subsequently, these numbers being substantially higher than those obtained by the same non-functionalized DAB G3 dendrimer. The dendron therefore makes it possible to produce bonds with the AAs, and participates greatly in a better encapsulation of the AAs at the core of the dendrimer, a property unknown at the start.

It therefore appears that this novel dendrimer associates all the advantages at once, namely good biocompatibility, good solubilization in water and also excellent transport efficiency of the AA, the last property being an additional advantage compared to the dendrimer functionalized with the MEAC monomer. c) The “Nona-Amine” Dendrimer Functionalized by the PFPTTEG Dendron

The PFPTTEG dendron is first synthesized according to example 2 in order to carry out the functionalization, according to the procedure described in example 4, of a novel water-soluble dendrimer containing nine branches, known here as the nona-amine dendrimer.

Procedure for Encapsulating Vitamin C:

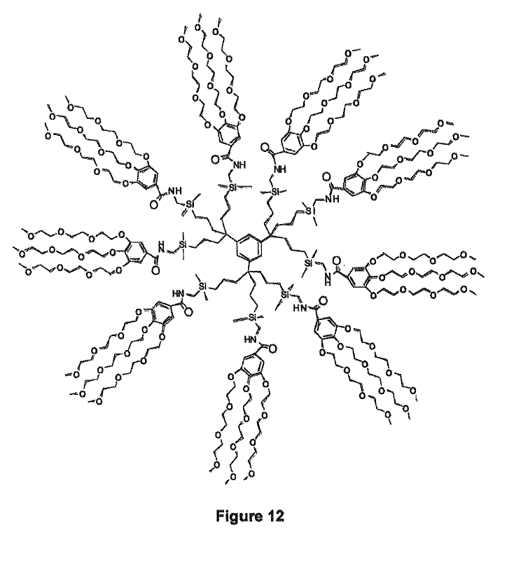

Fourteen samples (El to E14) comprising increasing amounts of ascorbic acid compared to the “nona-amine” dendrimer, functionalized by the PFPTTEG dendron (FIG. 12) were prepared as follows:
A solution S8 of ascorbic acid is obtained by dissolving 26.7 mg of ascorbic acid (AA) in 2 ml of D₂O.
Each sample E was prepared by adding a volume V of solution S8 of ascorbic acid into an NMR tube containing 5 mg of “nona-amine” dendrimer functionalized by the PFPTTEG dendron in 300 μl of D₂O.

The amounts of AA introduced and the ratio of the number of molecules of AA introduced per dendrimer into the NMR tube for each sample are listed in table 10 below.

TABLE 10

| | | | Ratio of the |
|---|---|---|---|
| | | | number of |
| | | | molecules of AA |
| | | | per DAB G3 |
| | Volume V of | Mass of AA | functionalized by |
| | solution S8 | introduced | the PFPTTEG |
| E | introduced (μL) | (mg) | dendron |
| 1 | 0 | 0 | 0 |
| 2 | 20 | 0.26 | 2 |
| 3 | 50 | 0.67 | 5 |
| 4 | 100 | 1.34 | 10 |
| 5 | 160 | 2.14 | 16 |
| 6 | 200 | 2.67 | 20 |
| 7 | 250 | 3.34 | 25 |
| 8 | 300 | 4.01 | 30 |
| 9 | 450 | 6.01 | 45 |
| 10 | 600 | 8.01 | 60 |
| 11 | 750 | 10.01 | 75 |
| 12 | 900 | 12.02 | 90 |
| 13 | 1000 | 13.35 | 100 |
| 14 | 1500 | 20.03 | 150 |

Titration of the Ascorbic Acid Contained in the Dendrimer and Results:

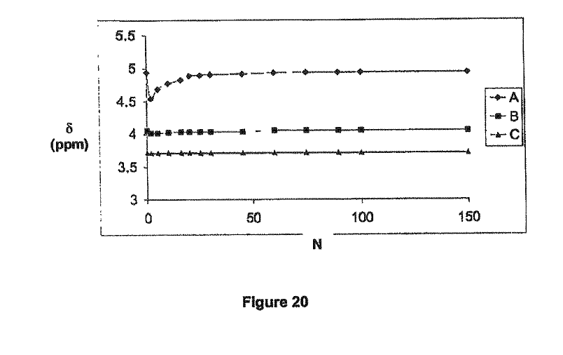

The NMR titration of the AA is carried out with this novel water-soluble functionalized dendrimer in order to identify the part played by the TEG chains in the transport of vitamin C. The results are given in the graph of the deshielding of the protons of AA presented in FIG. 20.

By virtue of this dendrimer functionalized with the PFPTTEG dendrons, it is possible to transport more than a hundred AAs.

Since this molecule comprises 27 TEG chains, the latter create electrostatic bonds and a steric conformation which are favorable to holding molecules of ascorbic acid within the molecule, thus allowing the transport thereof. This dendrimer, not being composed of any primary or tertiary amine, clearly demonstrates a chemical composition and a conformation that are ideal for the encapsulation of molecules of vitamin C by virtue of its particular properties provided by its core and its dendrons.

d) Conclusion

Ascorbic acid, the exogenous supply of which is essential for the human body, therefore has possibilities of being transported by certain organic molecules through a cosmetic product. Indeed, three types of vectors have been tested, namely pure commercial dendrimers, these same dendrimers functionalized with ethylene glycol chains, and also novel water-soluble dendrimers also comprising ethylene glycol chains, the synthesis of which is carried out entirely in the laboratory.

The dendrimers and their particular properties enable an excellent transport efficiency of molecules of ascorbic acid, especially when they are functionalized with a dendron comprising three triethylene glycol chains. Specifically, the latter create hydrogen bonds with the molecules of ascorbic acid and also enable them to be encapsulated at the core of the dendrimer, while providing properties of solubility in water (and also in a large number of solvents), of biocompatibility and above all of stability. Thus, the PFPTTEG dendron clearly participates in a better encapsulation of the molecules of vitamin C and also produces hydrogen bonds with these same molecules which can then be visualized by NMR. On the other hand, the chain of the MEAC group, three times shorter than that of the PFPTTEG group, does not make it possible to see, in NMR, a participation of the MEAC group in the encapsulation of the molecules of vitamin C.

The transport efficiencies of the novel water-soluble dendrimers functionalized with PEG chains differ depending on whether the dendron is more or less imposing with longer or shorter chains. The two dendrimers functionalized with the PFPTTEG dendron lead to a better transport efficiency; indeed, by virtue of their steric hindrance at the periphery of the dendrimers, they make it possible to keep the molecules of AA within the dendrimers, without however disturbing the passage of the molecules of AA to its core.

As regards the transport efficiency of molecules of vitamin C, the best performing dendrimer is that for which the synthesis is carried out entirely in the laboratory, namely the nona-amine functionalized by nine dendrons, i.e. 27 ethylene glycol chains. Furthermore, since this dendrimer does not contain internal amines, it should have a toxicity lower than those commercialized.

To conclude, the dendrimers appear to be suitable for the transport of molecules of ascorbic acid in cosmetics by virtue of their controlled multivalency which may be used to attach simultaneously one or more substances (medicaments, enzymes, medical imaging contrast agents, etc., here vitamin C), and also targeting and solubilizing groups at the periphery. Furthermore, this type of vector has the advantage of being able to be synthesized in various sizes by varying its generation. Finally, since dendrimers are molecules that have a well-defined structure (polymolecularity equal to one), they could provide a substance having reproducible pharmacokinetics, which is a certain advantage for the future application thereof.

### LIST OF REFERENCES

(1) Tomalia, D. A.; Baker, H.; Hall, M.; Kallos, G.; Martin, S.; Roeck, J.; Smith, P. Macromolecules (1986) 19, 2466.
(2) Astruc, D.; Research Avenues On Dendrimers towards Molecular Biology from Biomimation to Medicinal Engineering; C.R. Acad. Sci. (1996), 322, Serie 2b, 757-766.
(3) Vandamme, Th. F.; Brobeck, L.; J. Control. Release (2005), 102(1), 23-38.
(4) Zhuo R. X.; Du B.; Lu Z. R.; J. Control. Release (1999) 57, 249
(5) Liu, M.; Kono, K.; Fréchet, J. M. J.; J. Polym. Sci.: part A: Polym. Chem. (1999) 3492.
(6) Pan, Y.; Ford, W. T.; Amphiphilic Dendrimers with both octyl and triethyleneoxy methyl ether chain ends, Macromolecules (2000), 33, 3731-3738.
(7) Malik, N.; Wiwittanapatapee, R.; Klopsh, R.; Lorenz, K.; Frey, H.; Weener, J. W. et al, J. Control. Release (2000), 65, 133.
(8) Kojima, C.; Kono, K.; Maruyama, K.; Takagishi, T.; Synthesis of Polyamidoamine Dendrimers having Poly(Ethylene Glycol) Grafts and their Ability to Encapsulate Anticancer Drugs. Bioconjugate Chem. (2000) 11, 910-7.
(9) Baars, M. W. P. L. et al., The localization of guests in water-soluble Poly(propylene imine) dendrimers, Angew. Chem. Int. Ed. (2000), 39(7), 1285-1288.

1. A conjugated dendrimer comprising at least one water-soluble dendrimer encapsulating at least one molecule of vitamin C.

2. The conjugated dendrimer as claimed in claim 1, in which said, at least one, dendrimer has a symmetrical radial structure.

3. The conjugated dendrimer as claimed in claim 1, in which said, at least one, dendrimer has a globular structure.

4. The conjugated dendrimer as claimed in claim 1, in which said, at least one, dendrimer has a diameter of 10 Å to 50 Å.

5. The conjugated dendrimer as claimed in claim 1, in which said, at least one, dendrimer has at its periphery mainly free primary amine functional groups or ammonium functional groups.

6. The conjugated dendrimer as claimed in claim 1, in which said, at least one, dendrimer has one of the following structures:

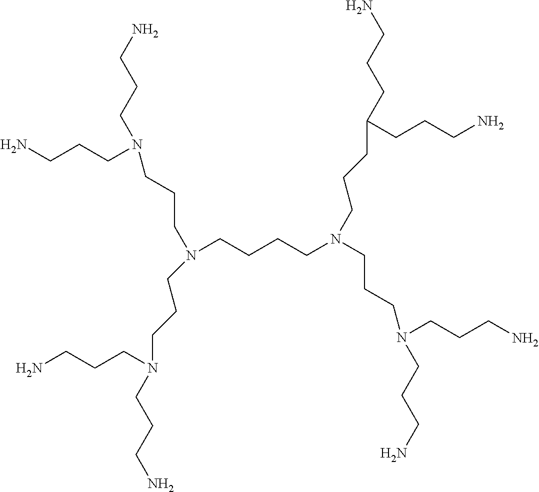

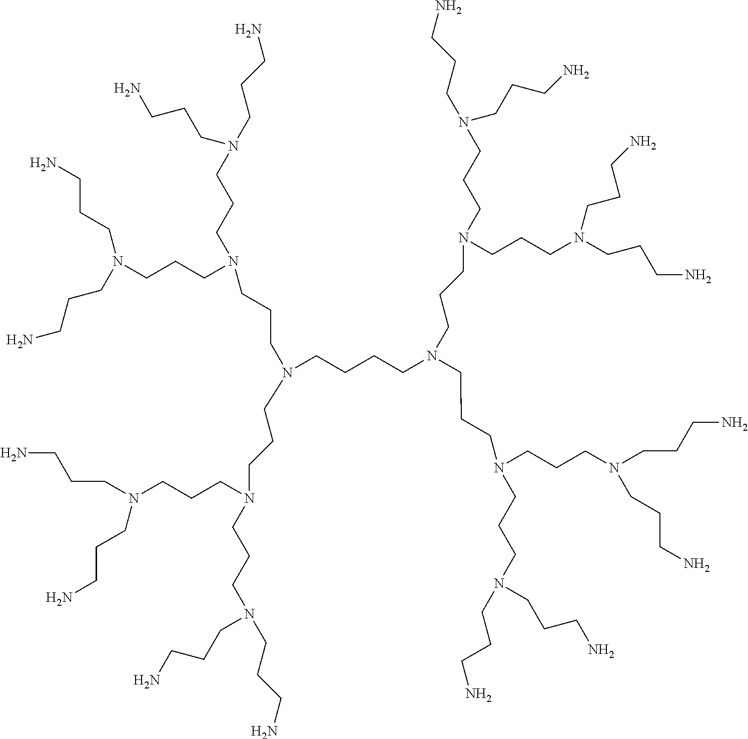

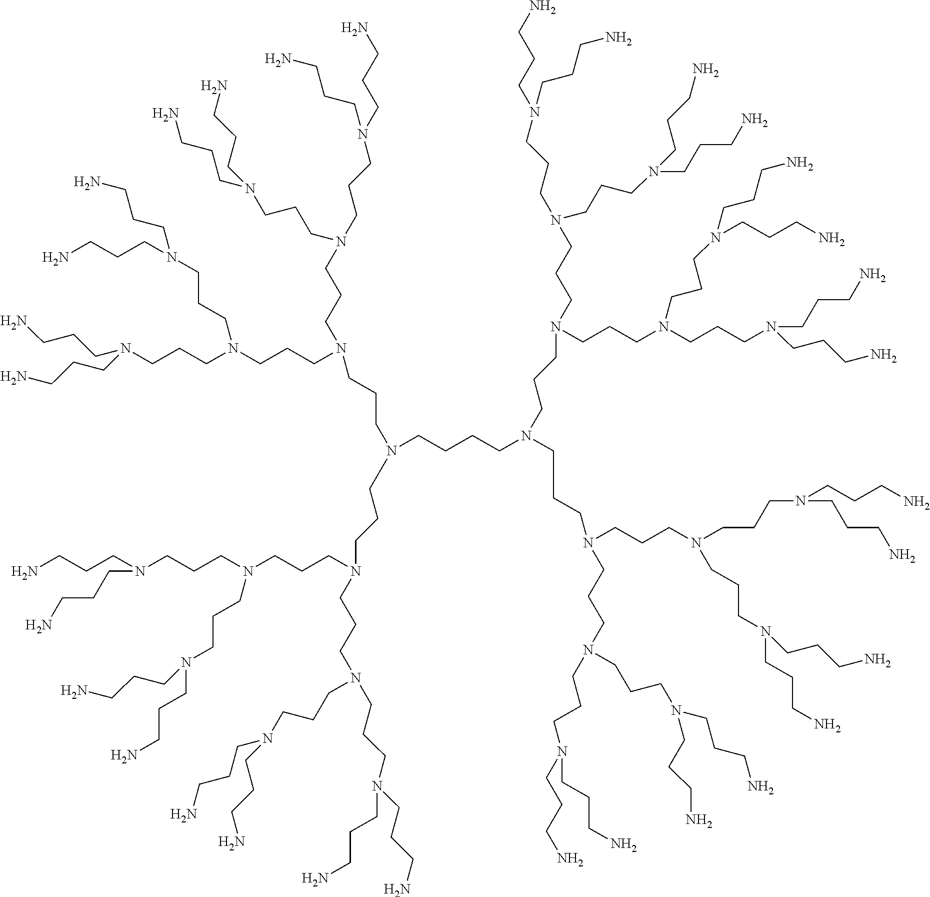

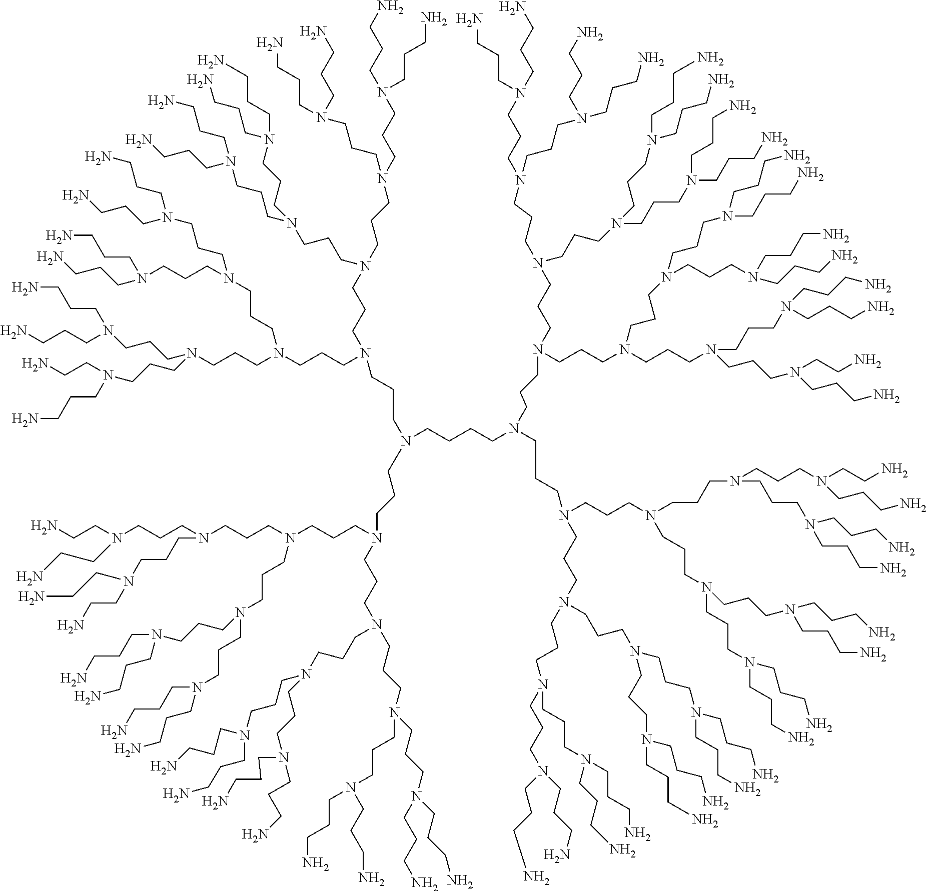

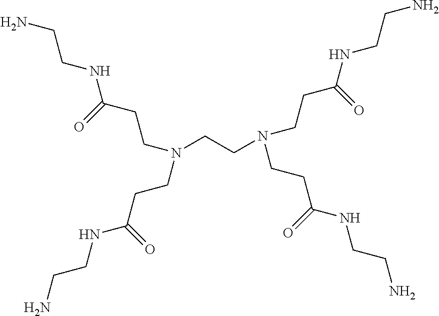

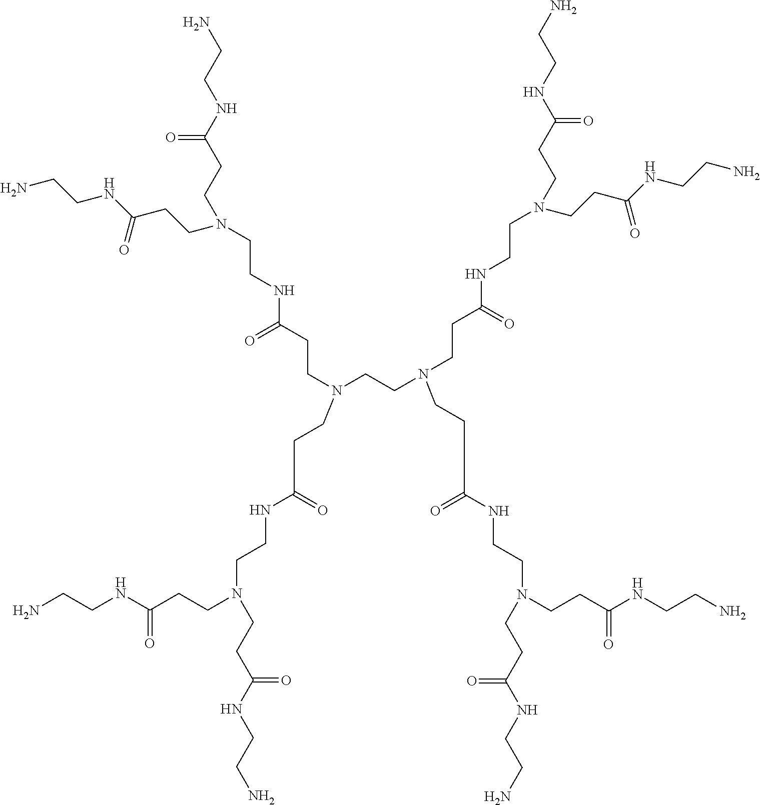

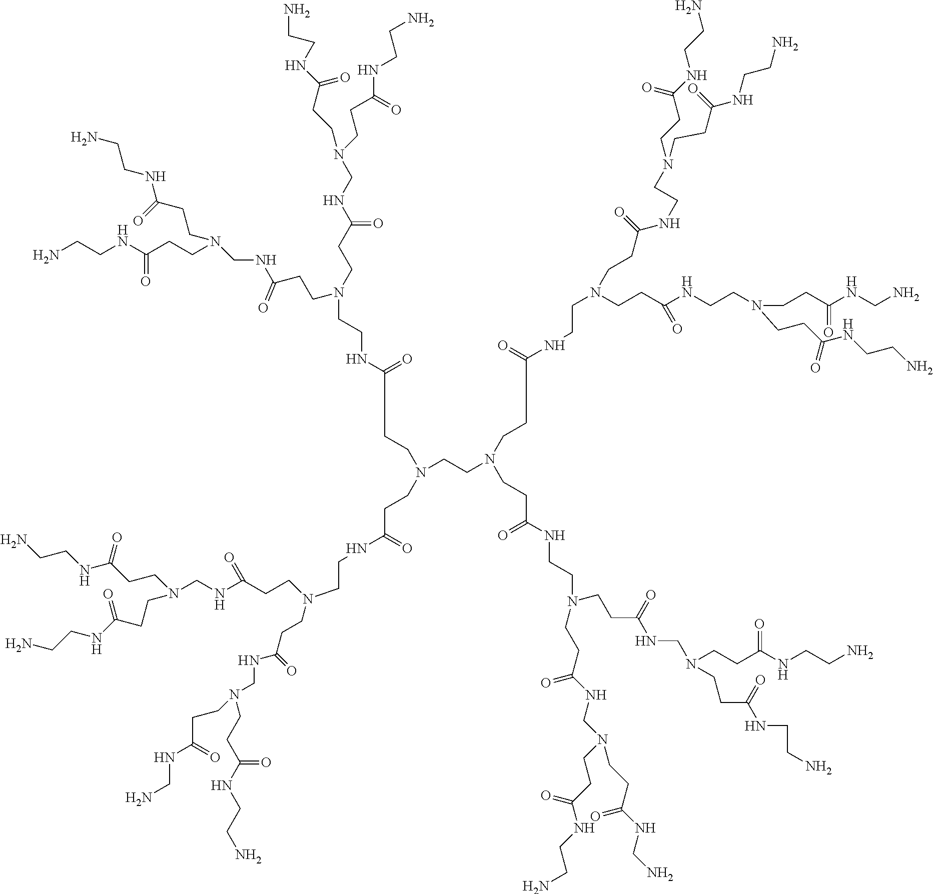

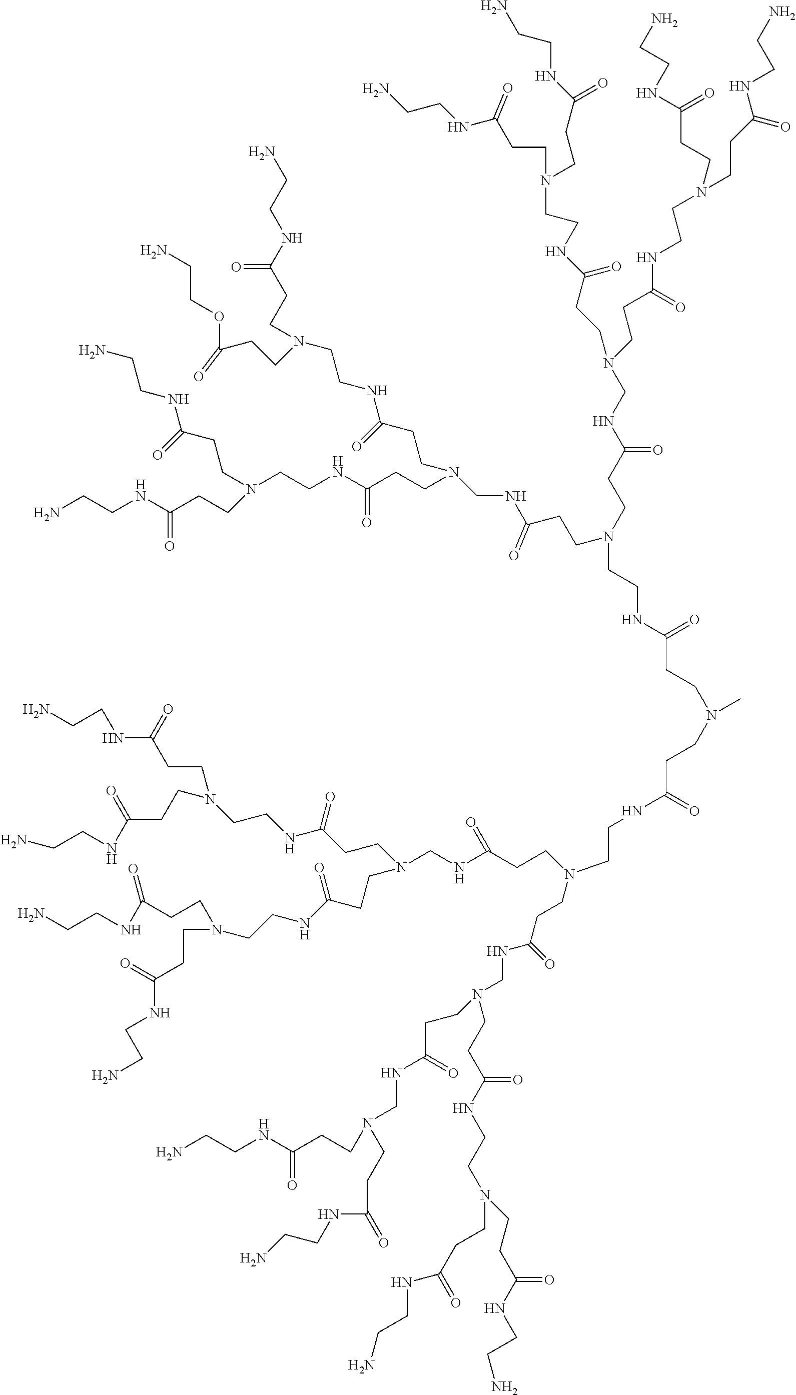

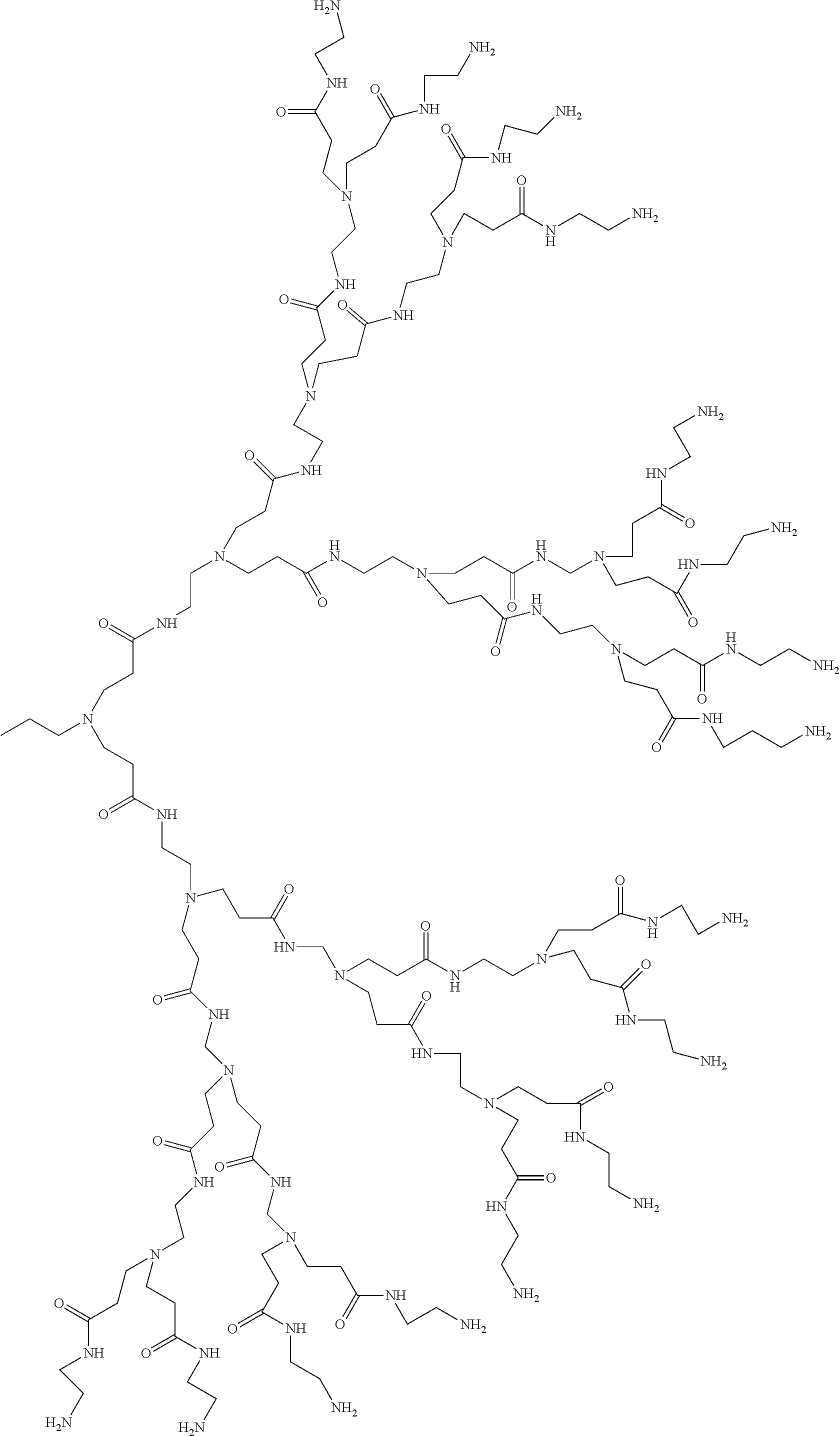

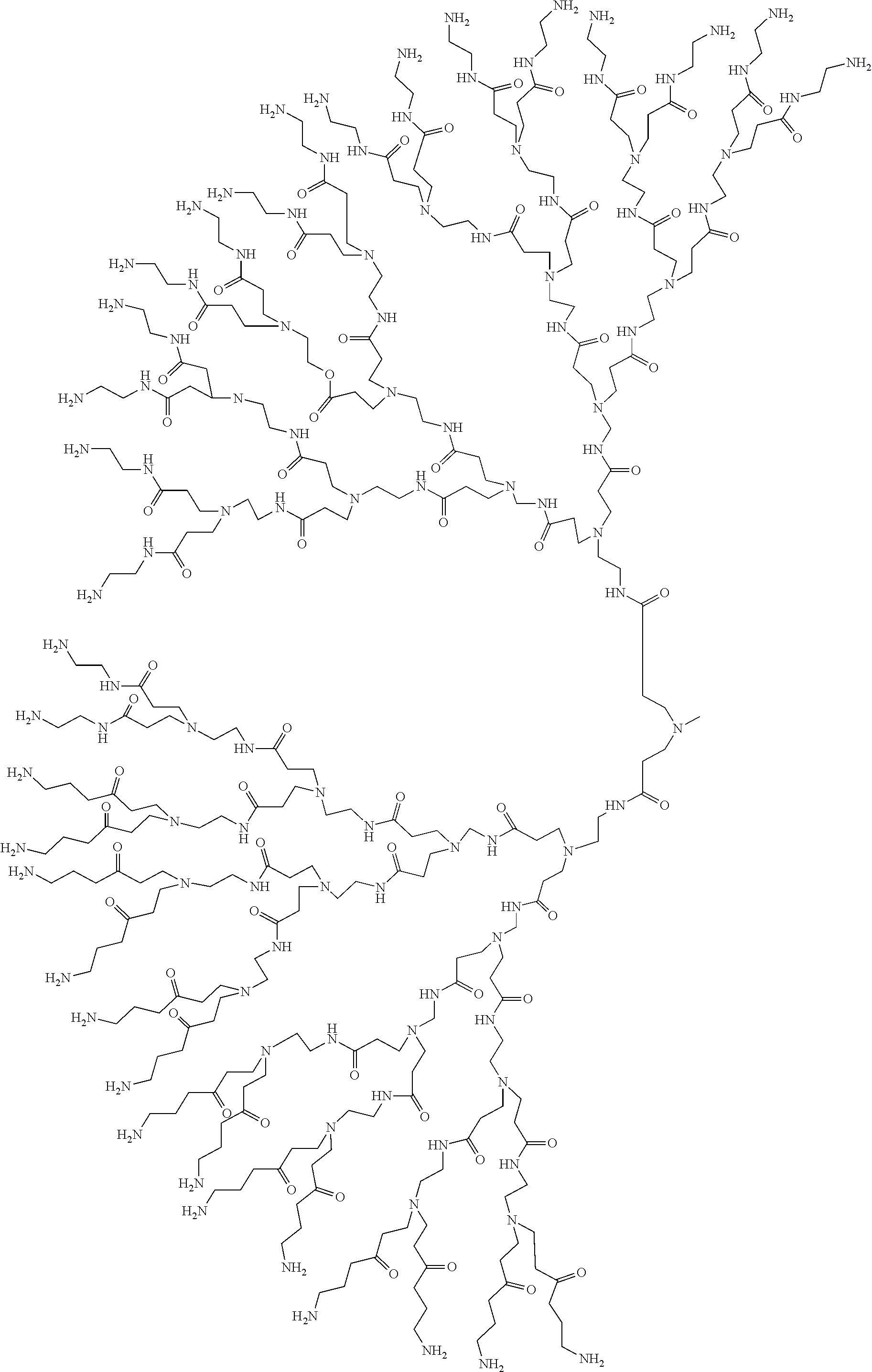

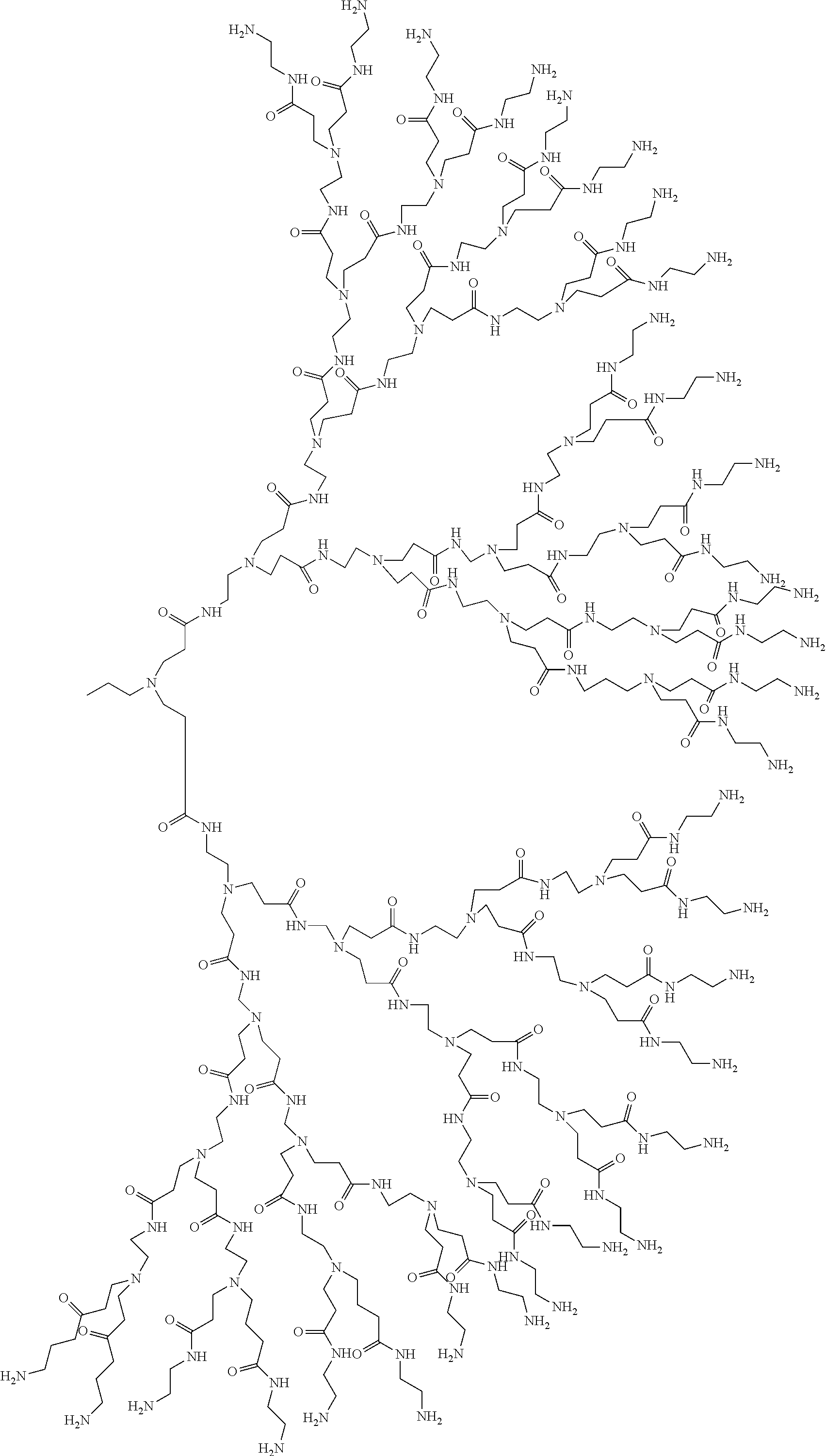

7. The conjugated dendrimer as claimed in claim 1, in which said, at least one, dendrimer is functionalized at its periphery with at least one organic surface agent.

8. The conjugated dendrimer as claimed in claim 7, in which said, at least one, organic surface agent is selected from the group comprising: a pharmaceutical molecule, a targeting molecule or a solubilizing group.

9. The conjugated dendrimer as claimed in claim 8, in which the organic surface agent is a solubilizing group having polyethylene glycol chains.

10. The conjugated dendrimer as claimed in claim 9, in which the solubilizing group corresponds to one of the structures (II) or (III) below: 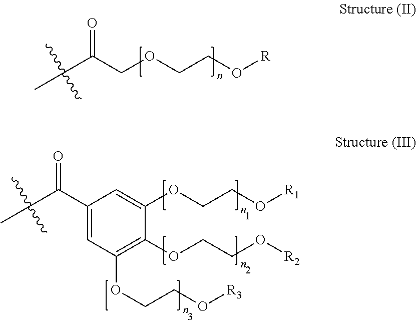
in which:
n, n₁, n₂ and n₃ independently represent an integer from 1 to 500; and
R, R₁, R₂ and R₃ independently represent a C₁ to C₆ alkyl, a C₂ to C₆ alkene, a C₂ to C₆ alkyne or a C₆ to C₁₀ aryl which is optionally substituted.

11. The conjugated dendrimer as claimed in claim 10, in which the solubilizing group corresponds to one of the structures (IV) or (V) below: 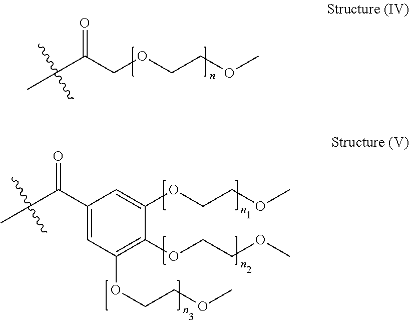
in which n represents 1 and n₁, n₂ and n₃ represent 3.

12. The conjugated dendrimer as claimed in claim 1, in which said, at least one, water-soluble dendrimer corresponds to the following structure: 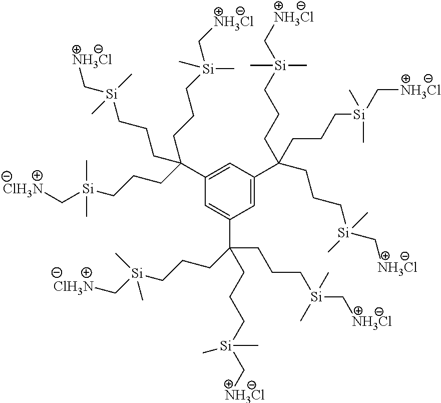
which dendrimer is functionalized at its periphery by a solubilizing group corresponding to one of the structures (II) or (III) below: 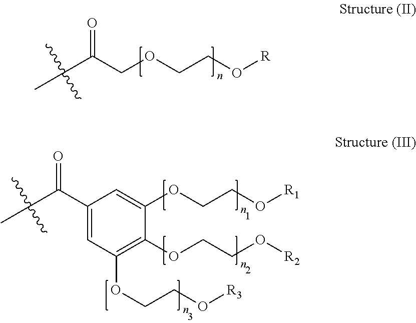
in which:
n, n₁, n₂ and n₃ independently represent an integer from 1 to 500; and
R, R₁, R₂ and R₃ independently represent a C₁ to C₆ alkyl, a C₂ to C₆ alkene, a C₂ to C₆ alkyne or a C₆ to C₁₀ aryl which is optionally substituted.

13. The conjugated dendrimer as claimed in claim 12, in which said, at least one, water-soluble dendrimer corresponds to the following structure:

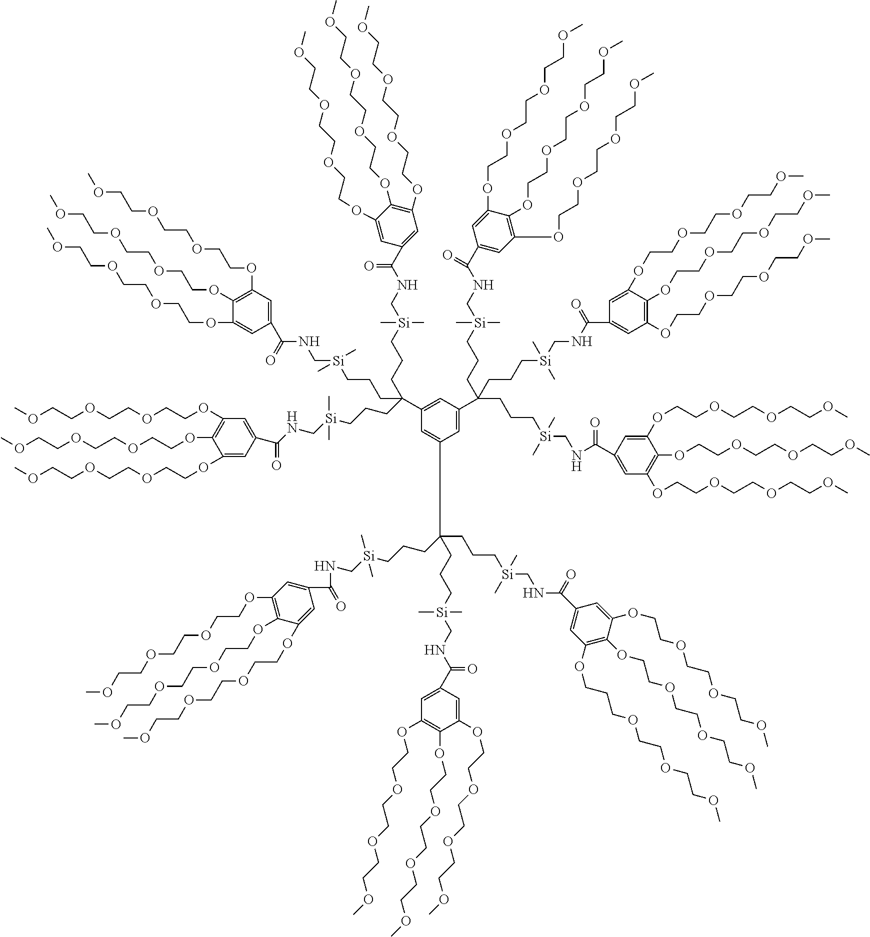

14. The conjugated dendrimer as claimed in claim 1, in which said, at least one, water-soluble dendrimer makes supramolecular bonds with the at least one molecule of vitamin C.

15. The conjugated dendrimer as claimed in claim 1, in which said, at least one, molecule of vitamin C is held at the core or at the periphery of the at least one water-soluble dendrimer.

16. The conjugated dendrimer as claimed in claim 1, in which the average encapsulation number is from 14 (±1) to 500 (±20) molecules of vitamin C per dendrimer.

17. A cosmetic composition comprising a conjugated dendrimer as claimed in claim 1.

18. The conjugated dendrimer as claimed in claim 1 as an antioxidant and/or anti-inflammatory agent.

19. The use of the conjugated dendrimer as claimed in claim 1, for the preparation of an anti-aging or anti-wrinkle cosmetic composition.

20. A cosmetic care method comprising a step that consists in applying a cosmetic composition comprising a conjugated dendrimer as claimed in claim 1 to the skin.

21. A method of preparing a conjugated dendrimer comprising at least one water-soluble dendrimer that encapsulates at least one molecule of vitamin C, said method comprising a step of reaction of said water-soluble dendrimer with said at least one molecule of vitamin C in a solvent and at a temperature which facilitate the formation of supramolecular bonds of the dendrimer and of the molecule of vitamin C.

22. The method as claimed in claim 21, in which the association of the dendrimer and of the molecule of vitamin C is carried out by means of at least one of the following bonds: formation of ammonium ascorbate with an amine functional group of the dendrimer, formation of a hydrogen bond between a hydroxyl functional group of the vitamin C and an amine functional group of the dendrimer.

23. The method as claimed in claim 21, comprising thereaction, in water, of a sufficient amount of vitamin C to obtain a concentration of less than 10² mg/ml, with a sufficient amount of dendrimer to obtain a conjugated dendrimer having a vitamin C/dendrimer ratio of 14 to 500, the reaction being carried out at a pH of 6 to 8 and at a temperature of 25° C.±5° C.

24. The method as claimed in claim 21, comprising, in addition, a step of functionalization, before the step of reaction with the vitamin C, which consists in functionalizing the periphery of the dendrimer with an organic surface agent selected from the group comprising: a pharmaceutical molecule, a targeting molecule or a solubilizing group.

25. The method as claimed in claim 22, comprising thereaction, in water, of a sufficient amount of vitamin C to obtain a concentration of less than 10² mg/ml, with a sufficient amount of dendrimer to obtain a conjugated dendrimer having a vitamin C/dendrimer ratio of 14 to 500, the reaction being carried out at a pH of 6 to 8 and at a temperature of 25° C.±5° C.

26. The method as claimed in claim 22, comprising, in addition, a step of functionalization, before the step of reaction with the vitamin C, which consists in functionalizing the periphery of the dendrimer with an organic surface agent selected from the group comprising: a pharmaceutical molecule, a targeting molecule or a solubilizing group.

27. The method as claimed in claim 23, comprising thereaction, in water, of a sufficient amount of vitamin C to obtain a concentration of less than 10² mg/ml, with a sufficient amount of dendrimer to obtain a conjugated dendrimer having a vitamin C/dendrimer ratio of 14 to 500, the reaction being carried out at a pH of 6 to 8 and at a temperature of 25° C.±5° C.
